SEQUENCE LISTING (19 sequences)

SEQ ID NO: 1 - RNA, 59 nt, Artificial sequence
  Synthetic oligonucleotide
ggugauaccagcaucgucuugaugcccuuggcagcaccnnnnnnnnnnncaacaagaug

SEQ ID NO: 2 - RNA, 59 nt, Artificial sequence
  Synthetic oligonucleotide
ggugauaccagcaucgucuugaugcccuuggcagcacccugagaaggggcaacaagaug

SEQ ID NO: 3 - RNA, 65 nt, Artificial sequence
  Synthetic oligonucleotide
ggugauaccagcaucgucuugaugcccuuggcagcacccgcugcnnagggggunncaacaagaug

SEQ ID NO: 4 - RNA, 65 nt, Artificial sequence
  Synthetic oligonucleotide
ggugauaccagcaucgucuugaugcccuuggcagcacccgcugcgcaggggguaucaacaagaug

SEQ ID NO: 5 - RNA, 64 nt, Artificial sequence
  Synthetic oligonucleotide
nnnnnnnnnnnnggugauaccagcaucgucuugaugcccuuggcagcaccaagacaacaagaug

SEQ ID NO: 6 - RNA, 69 nt, Artificial sequence
  Synthetic oligonucleotide
gguaccugauaagauagggugauaccagcaucgucuugaugcccuuggcagcaccaagacaacaagaug

SEQ ID NO: 7 - RNA, 79 nt, Artificial sequence
  Synthetic oligonucleotide
cacuauagguaccugauaagauaggggugauaccagcaucgucuugaugcccuuggcagcaccaagggacaacaagaug

SEQ ID NO: 8 - RNA, 60 nt, Artificial sequence
  Synthetic oligonucleotide
ggugauaccagcaucgucuugaugcccuuggcagcacccugcuaagguaacaacaagaug

SEQ ID NO: 9 - RNA, 63 nt, Artificial sequence
  Synthetic oligonucleotide
ggugauaccagcaucgucuugaugcccuuggcagcacccugcuaaggagguaacaacaagaug

SEQ ID NO: 10 - DNA, 15 bp, Artificial sequence
  Synthetic oligonucleotide
atacgactcactata

SEQ ID NO: 11 - RNA, 65 nt, Artificial sequence
  Synthetic oligonucleotide
gguaccggugauaccagcaucgucuugaugcccuuggcagcacccugagaaggggcaacaagaug

SEQ ID NO: 12 - RNA, 71 nt, Artificial sequence
  Synthetic oligonucleotide
gguaccggugauaccagcaucgucuugaugcccuuggcagcacccgcugcgcaggggguaucaacaagaug

SEQ ID NO: 13 - RNA, 72 nt, Artificial sequence
  Synthetic oligonucleotide
gguaccugauaagauaggggugauaccagcaucgucuugaugcccuuggcagcaccaagggacaacaagaug

SEQ ID NO: 14 - RNA, 66 nt, Artificial sequence
  Synthetic oligonucleotide
gguaccggugauaccagcaucgucuugaugcccuuggcagcacccugcuaagguaacaacaagaug

SEQ ID NO: 15 - RNA, 69 nt, Artificial sequence
  Synthetic oligonucleotide
gguaccggugauaccagcaucgucuugaugcccuuggcagcacccugcuaaggagguaacaacaagaug

SEQ ID NO: 16 - RNA, 66 nt, Artificial sequence
  Synthetic oligonucleotide
gguaccggugauaccagcaucgucuugaugcccuuggcagcacccugcuaaggaggcaacaagaug

SEQ ID NO: 17 - RNA, 60 nt, Artificial sequence
  Synthetic oligonucleotide
ggugauaccagcaucgucuugaugcccuuggcagcacccugcuaaggaggcaacaagaug

SEQ ID NO: 18 - RNA, 38 nt, Artificial sequence
  Synthetic oligonucleotide
ggugauaccagcaucgucuugaugcccuuggcagcacc

SEQ ID NO: 19 - RNA, 15 nt, Artificial sequence
  Synthetic oligonucleotide
auacgacucacuaua

(19) United States
(12) Patent Application Publication
(10) Pub. No.: US 2012/0244601 A1
(43) Pub. Date: Sep. 27, 2012

(54) RIBOSWITCH BASED INDUCIBLE GENE EXPRESSION PLATFORM

(76) Inventors: Carolyn R. Bertozzi, Berkeley, CA (US); Justin P. Gallivan, Atlanta, GA (US); Jessica C. Seeliger, Berkeley, CA (US); Shana Topp, Berkeley, CA (US)

(21) Appl. No.: 13/423,896

(22) Filed: Mar. 19, 2012

Related U.S. Application Data
(60) Provisional application No. 61/466,347, filed on Mar. 22, 2011.

(51) Int. Cl. C12N 1/21; C12N 15/70; C07H 21/04

(52) U.S. Cl. 435/252.3; 536/24.1; 435/320.1

(57) ABSTRACT

The present disclosure provides a synthetic translation regulator, as well as gene expression cassettes and gene expression constructs comprising the synthetic translation regulator. The present disclosure further provides genetically modified bacterial host cells comprising a subject synthetic translation regulator; and methods of regulating gene expression in such host cells.

This application claims the benefit of U.S. Provisional Patent Application No. 61/466,347 filed Mar. 22, 2011, which application is incorporated herein by reference in its entirety.

### STATEMENT REGARDING FEDERALLY SPONSORED RESEARCH

This invention was made with government support under Grant Nos. AI051622 and GM074070 awarded by The National Institutes of Health. The government has certain rights in the invention.

### BACKGROUND

The ability to precisely control gene expression has greatly enhanced the study of microbial genetics and behavior. Common model systems, such as Escherichia coli, typically have a variety of genetic control elements available, such as the isopropylthiol-β-D-galactoside (IPTG)-inducible lac operon and the arabinose-inducible araC promoter. However, for many bacteria, simple-to-use methods for inducing gene expression in a ligand-dependent fashion do not exist. While it is possible in principle to transfer the inducible regulatory machinery from one species to another, issues including promoter usage, protein folding, and ligand permeability present challenging obstacles. Indeed, with the exception of the tetracycline-inducible expression system most protein-based ligand-inducible expression systems that function well in E. coli have proven difficult to transport into a broad range of bacteria.

Bacterial riboswitch RNAs are genetic control elements that are located primarily within the 5′-untranslated region (5′-UTR) of the main coding region of a particular mRNA. Studies of riboswitches indicate that riboswitch elements generally include two domains: a natural aptamer that serves as the ligand-binding domain, and an “expression platform” that interfaces with RNA elements that are involved in transcription and/or translation.

There is a need in the art for means of controlling gene expression in a variety of bacterial species.

### LITERATURE

U.S. Patent Publication No. 2010/0286082; Bayer and Smolke (2005) Nat. Biotech. 23:337; Desai and Gallivan (2004) J. Am. Chem. Soc. 126:13247; Lynch et al. (2007) Chem. Biol. 14:173; Lynch et al. (2009) Nucl. Acids. Res. 37:184; Suess et al. (2004) Nucl. Acids Res. 32:1610; Weigand et al. (2008) RNA 14:89; and Werstuck and Green (1998) Science 282:296; U.S. Pat. No. 7,563,601.

### SUMMARY

The present disclosure provides a synthetic translation regulator, as well as gene expression cassettes and gene expression constructs comprising the synthetic translation regulator. The present disclosure further provides genetically modified bacterial host cells comprising a subject synthetic translation regulator; and methods of regulating gene expression in such host cells.

### BRIEF DESCRIPTION OF THE DRAWINGS

FIGS. 1A and 1B depict development of Riboswitch A. FIG. 1A depicts SEQ ID NO:1; FIG. 1B depicts SEQ ID NO:2.

FIGS. 2A and 2B depict development of Riboswitch B. FIG. 2A depicts SEQ ID NO:3; FIG. 2B depicts SEQ ID NO:4.

FIGS. 3A-C depict development of Riboswitch C. FIG. 3A depicts SEQ ID NO:5; FIG. 3B depicts SEQ ID NO:6; FIG. 3C depicts SEQ ID NO:7.

FIGS. 4A-C depict development of Riboswitches D and E. FIG. 4A depicts SEQ ID NO:1; FIG. 4B depicts SEQ ID NO:8; FIG. 4C depicts SEQ ID NO:9.

FIGS. 5A and 5B depict dose response profiles in E. coli.

FIGS. 6A and 6B depict dose response profiles in Acinetobacter baylyi.

FIGS. 7A and 7B depict dose response profiles in Bacillus subtilis.

FIGS. 8A and 8B depict dose response profiles in Acinetobacter baumannii.

FIG. 9 depicts a dose response profile in Agrobacterium tumefaciens.

FIGS. 10A and 10B depict dose response profiles in Mycobacterium smegmatis.

FIGS. 11A and 11B depict dose response profiles in Streptococcus pyogenes.

FIGS. 12A and 12B depict a comparison of A. tumefaciens and Mycobacterium magneticum 16S rRNA pairing with the ribosome binding site of Riboswitch A. FIGS. 12A and 12B depict SEQ ID NO:2.

FIG. 13 provides a schematic depiction of a riboswitch.

FIG. 13 depicts SEQ ID NO:10

FIGS. 14A and 14B depict bacterial phylogeny (FIG. 14A). Species studied here are shown in the ovals. FIG. 14B depicts activation ratios and expression levels for riboswitches A-E in each organism.

FIG. 15 depicts images of riboswitches B and C controlling the expression of a MamK-GFP fusion in M. magneticum in the presence of theophylline (1 mM) as a function of time.

FIG. 16 provides nucleotide sequences of riboswitches A-E*, which are also provided in Table 1. Sequences are designated as follows: A=SEQ ID NO:11; B=SEQ ID NO:12; C±SEQ ID NO:13; D=SEQ ID NO:14; E=SEQ ID NO:15; E*=SEQ ID NO:16.

FIG. 17 depicts design and applications of the riboswitch-based gene regulation platform for mycobacteria.

FIG. 17 depicts SEQ ID NO:17.

FIGS. 18A-C depict verification of theophylline-induced gene regulation in M. smegmatis (Msmeg) and M. tuberculosis (Mtb).

FIG. 19 depicts demonstration of riboswitch-controlled Mtb gene expression in a macrophage infection model.

FIG. 20 provides Table 2.

FIG. 21 depicts a comparison between P_{gs}-derived riboswitch expression systems and those derived from Pₕₛₚ₆₀.

FIGS. 22A and 22B depict theophylline-dependent induction of GFP or β-galactosidase expression.

FIG. 23 depicts M. smegmatis growth as a function of theophylline concentration.

FIG. 24 depicts inducible Mtb gene expression in a macrophage infection model from a riboswitch based on the glutamine synthase promoter P_{gs}.

### DEFINITIONS

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

The term “aptamer” as used herein refers to a fragment (or a domain) of nucleic acid that selectively binds to a ligand or molecule. The introduction of a ligand to a ligand-specific aptamer causes conformational changes within the aptamer and influences nucleic acids adjacent to the aptamer.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems.

A “host cell,” as used herein, denotes an in vivo or in vitro prokaryotic cell, which prokaryotic cells can be, or have been, used as recipients for a nucleic acid (e.g., a subject gene expression construct), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., a subject expression vector. For example, a subject prokaryotic host cell is a genetically modified prokaryotic host cell (e.g., a bacterium), by virtue of introduction into a suitable prokaryotic host cell a heterologous nucleic acid, e.g., an exogenous nucleic acid that is foreign to (not normally found in nature in) the prokaryotic host cell, or a recombinant nucleic acid that is not normally found in the prokaryotic host cell.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a synthetic translation regulator” includes a plurality of such synthetic translation regulators and reference to “the genetically modified bacterium” includes reference to one or more genetically modified bacteria and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a synthetic translation regulator, as well as gene expression cassettes and gene expression constructs comprising the synthetic translation regulator. The present disclosure further provides genetically modified bacterial host cells comprising a subject synthetic translation regulator; and methods of regulating gene expression in such host cells.

### Synthetic Translation Regulator

The present disclosure provides a synthetic translation regulator, for use in regulating translation of an operably linked coding region in a wide variety of bacterial cells. A subject synthetic translation regulator is also referred to herein as a “theophylline-responsive riboswitch” or, simply, a “riboswitch.”

A subject synthetic translation regulator comprises, in order from 5′ to 3′: a) a theophylline-binding aptamer; b) a nucleic acid (a “first nucleic acid linker”) of from 0 to 20 nucleotides (e.g., where the first nucleic acid linker, if present, has a length of 1, 2, 3, 4, 5, 6, 7, 8, 9, 10, 11, 12, 13, 14, 15, 16, 17, 18, 19, or 20 nucleotides); c) a ribosome binding site; and d) a nucleic acid (a “second nucleic acid linker”) of from 0 to 20 nucleotides (e.g., where the second nucleic acid linker, if present, has a length of 1, 2, 3, 4, 5, 6, 7, 8, 9, 10, 11, 12, 13, 14, 15, 16, 17, 18, 19, or 20 nucleotides).

In some embodiments, the theophylline-binding aptamer comprises the nucleotide sequence: 5′-GGUGAUACCAGCAUCGUCUUGAUGCCCUUGGCAGCACC-3′ (SEQ ID NO:18); and has a length of from about 38 nucleotides to about 40 nucleotides.

In some embodiments, the ribosome binding site (RBS) has a length of from 4 nucleotides to 10 nucleotides, and comprises the sequence AAGG. Exemplary RBS sequences include, but are not limited to, AGGGGGU, AAGGGG, AAGGG, AAGGU, AAGGAGGU, and AAGGAGG. The RBS provides for binding to 16S ribosomal RNA.

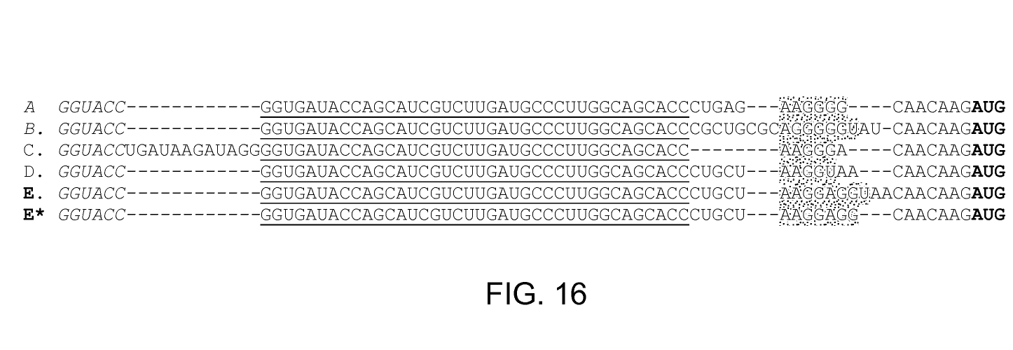

For example, a subject synthetic translation regulator can comprise a nucleotide sequence as depicted in FIG. 16 or Table 1 (in the Examples).

A subject synthetic translation regulator confers theophylline responsiveness on an operably linked coding region, when present in a bacterial cell. For example, in the absence of theophylline, translation of an operably linked coding region is at background levels, e.g., undetectable levels. In the presence of theophylline (e.g., from about 1 mM theophylline to about 2 mM theophylline), translation of the operably linked coding region is increased by from about 2-fold to about 100-fold, or more than 100-fold compared to the level of translation of the operably coding region in the absence of theophylline. For example, in the presence of theophylline, translation of the operably linked coding region is increased by from about 2-fold to about 5-fold, from about 5-fold to about 10-fold, from about 10-fold to about 25-fold, from about 25-fold to about 50-fold, or from about 50-fold to about 100-fold, or more than 100-fold, compared to the level of translation of the operably coding region in the absence of theophylline.

The present disclosure provides a nucleic acid comprising a subject synthetic translation regulator. A subject synthetic translation regulator can have a length of from about 50 nucleotides (nt) to about 100 nt, e.g., from about 50 nt to about 55 nt, from about 55 nt to about 60 nt, from about 60 nt to about 65 nt, from about 65 nt to about 70 nt, from about 70 nt to about 75 nt, or from about 75 nt to about 100 nt. A nucleic acid comprising a subject synthetic translation regulator can have a length of from about 50 nucleotides (nt) to about 100 nt (e.g., from about 50 nt to about 55 nt, from about 55 nt to about 60 nt, from about 60 nt to about 65 nt, from about 65 nt to about 70 nt, from about 70 nt to about 75 nt, or from about 75 nt to about 100 nt), and can have additional sequences 5′ and 3′ of the synthetic translation regulator, making the total length of the nucleic acid from about 50 nt to several kilobases (kb), e.g., 50 nt to about 100 nt, from about 100 nt to about 250 nt, from about 250 nt to about 500 nt, from about 500 nt to about 1 kb, from about 1 kb to about 2 kb, from about 2 kb to about 5 kb, or from about 5 kb to about 10 kb, or longer than 10 kb. A subject nucleic acid comprising subject synthetic translation regulator can be, e.g., a gene expression cassette, a gene expression construct, etc.

A subject nucleic acid can comprise two or more riboswitches. In some embodiments, the two or more riboswitches can be a subject theophylline-responsive riboswitch. In other embodiments, the two or more riboswitches can include one or more of a subject theophylline-responsive riboswitch; and one or more of a riboswitch that responds to a small molecule other than theophylline. For example, in some embodiments, a subject nucleic acid can comprise one or more of a subject theophylline-responsive riboswitch; and one or more of a riboswitch selected from a cyclic di-GMP-responsive riboswitch, an S-adenosylhomocysteine-responsive riboswitch, a preQ₁-responsive riboswitch, a Moco-responsive riboswitch, a SAM-responsive riboswitch, and a riboswitch as described in U.S. Patent Publication No. 2010/0286082.

A subject synthetic translation regulator can be used to control translation of an operably linked coding region (e.g., a coding region encoding a polypeptide of interest). A subject synthetic translation regulator can be used in research applications, e.g., to study regulation of a gene or genes. A subject synthetic translation regulator can be used to produce a polypeptide encoded by a coding region of interest, e.g., to control production of the polypeptide. A subject synthetic translation regulator can be used to control a conditional gene knockout.

A subject synthetic translation regulator can be generated using standard recombinant DNA techniques, or can be chemically synthesized using well-established methods.

### Gene Expression Cassettes

The present disclosure provides synthetic gene expression cassettes, which can be inserted into a wide variety of expression vectors (e.g., plasmids). In some embodiments, a subject gene expression cassette comprises, in order from 5′ to 3′ and in operable linkage: a) a promoter suitable for use in a prokaryotic host cell; b) a 5′ untranslated region (5′-UTR); and c) a subject synthetic translation regulator. The synthetic translation regulator can confer theophylline-regulatable translation of an operably linked coding region.

In some embodiments, a subject gene expression cassette further comprises a coding region. Thus, in some embodiments, a subject gene expression cassette comprises, in order from 5′ to 3′ and in operable linkage: a) a promoter suitable for use in a prokaryotic host cell; b) a 5′ untranslated region (5′-UTR); c) a subject synthetic translation regulator; and d) a coding region. The coding region typically comprises, at its 5′ terminus, an ATG start codon.

In some embodiments, the 5′-UTR comprises the sequence 5′-ATACGACTCACTATA-3′ (SEQ ID NO:10).

A suitable promoter includes one that is active in a bacterial host cell. Suitable promoters include inducible promoters and constitutive promoters. Suitable inducible promoters include, but are not limited to, promoters induced by, e.g., radiation, pH change, temperature change, alcohol, antibiotic, steroid, metal, salicylic acid, ethylene, benzothiadiazole, or other inducer compound. Exemplary inducers include, e.g., arabinose, lactose, maltose, sucrose, glucose, xylose, galactose, rhamnose, fructose, melibiose, starch, inulin, lipopolysaccharide, arsenic, cadmium, chromium, temperature, light, antibiotic, oxygen level, xylan, nisin, L-arabinose, allolactose, D-glucose, D-xylose, D-galactose, ampicillin, tetracycline, penicillin, pristinamycin, retinoic acid, and interferon. The promoter can be a naturally-occurring promoter or a synthetic (e.g., non-naturally-occurring; generated using recombinant means or chemically synthesized) promoter.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a lacUV5 promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol., 1991: 173(1): 86-93; Alpuche-Aranda et al., PNAS, 1992; 89(21): 10079-83), a nirB promoter (Harborne et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035; and the like.

Inducible promoters are well known in the art. Suitable inducible promoters include, but are not limited to, the pL of bacteriophage λ; Plac; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (see, e.g., Guzman et al. (1995) J. Bacteriol. 177:4121-4130); a xylose-inducible promoter, e.g., Pxyl (see, e.g., Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; a heat-inducible promoter, e.g., heat inducible lambda P_{L} promoter, a promoter controlled by a heat-sensitive repressor (e.g., CI857-repressed lambda-based expression vectors; see, e.g., Hoffmann et al. (1999) FEMS Microbiol Lett. 177(2):327-34); and the like.

Also suitable for use is a neutral, base or acid inducible promoter. Examples of acid inducible promoters include, but are not limited to HVA1 promoter (plant cells), P170, P1, or P3 (Lactococcus), baiA1, baiA3 (Eubacteria), lipF promoter (Mycobacteria), F₁F₀-ATPase promoter (Lactobacillus, Streptococcus, or Enterococcus), gadC, gad D (Lactococcus, Shignella), glutamate decarboxylase promoter (Mycobacteria, Clostridium, Listeria, Lactobacillus), or similar operons. See, for example, Cotter and Hill, Microbiol. and Mol. Biol. Rev. vol. 67, no. 3, pp. 429-453 (2003); Hagenbeek, et al., Plant Phys., vol. 123, pp. 1553-1560 (2000); Madsen, et al., Abstract, Mol. Microbiol. vol. 56, no. 3, pp. 735-746 (2005); U.S. Pat. No. 6,242,194; Richter, et al., Abstract, Gene, vol. 395, no. 1-2, pp. 22-28 (2007), Mallonee, et al., J. Bacteriol., vol. 172, no. 12, pp. 7011-7019 (1990). Examples of base inducible promoters include, but are not limited to, alkaline phosphatase promoters.

Where a subject gene expression cassette comprises a coding region, the coding region can encode any of a variety of polypeptides, without limitation. Non-limiting examples include, e.g., therapeutic polypeptides; polypeptides that produce, directly or indirectly, a detectable signal (e.g., a chromogen; a fluorophore; a luminogen, an enzyme that generates a product that produces a detectable signal; and the like); regulatory polypeptides (e.g., transcription factors); structural polypeptides; enzymes; and the like.

### Gene Expression Construct

The present disclosure provides a recombinant gene expression construct. A subject gene expression construct comprises a subject gene expression cassette, as described above, inserted into a vector suitable for use in a prokaryotic host cell.

In some embodiments, a subject recombinant gene expression construct comprises, in order from 5′ to 3′ and in operable linkage: a) a promoter suitable for use in a prokaryotic host cell; b) a 5′ untranslated region (5′-UTR); and c) a subject synthetic translation regulator. A coding region can be inserted 3′ of the synthetic translation regulator. For example, a subject recombinant gene expression construct can include a multiple cloning site 3′ of the synthetic translation regulator, for insertion of a coding region. The multiple cloning site can include two or more restriction endonuclease recognition sites, for ease of cloning. The two or more restriction endonuclease recognition sites can be provided in tandem, or can be overlapping.

In some embodiments, a gene expression construct further comprises a coding region. Thus, in some embodiments, a subject gene expression construct comprises, in order from 5′ to 3′ and in operable linkage: a) a promoter suitable for use in a prokaryotic host cell; b) a 5′ untranslated region (5′-UTR); c) a subject synthetic translation regulator; and d) a coding region. The coding region typically comprises, at its 5′ terminus, an ATG start codon.

Vectors suitable for use in prokaryotic host cells generally include an origin of replication, and can include, e.g., a selectable marker. Any of several well-known selectable markers such as neomycin resistance, ampicillin resistance, tetracycline resistance, chloramphenicol resistance, kanamycin resistance, and the like, can be used.

A wide variety of bacterial expression vectors are suitable for use, and can be modified by insertion of a subject gene expression cassette. Non-limiting examples include, e.g., pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). A broad host range vector such as pBAV1K can be used. However, any other plasmid or other vector may be used so long as it is compatible with the host cell.

In some embodiments, the vector is a high copy number plasmid. In other embodiments, the vector is a medium copy number plasmid. In still other embodiments, the vector is a low copy number plasmid. In some embodiments, the vector is maintained extrachromosomally. In other embodiments, the vector integrates into the genome of the host cell.

### Genetically Modified Prokaryotic Host Cells

The present disclosure provides a genetically modified prokaryotic host cell, where the prokaryotic host cell is genetically modified by introduction of a nucleic acid comprising a subject synthetic translation regulator, a nucleic acid comprising a subject gene expression cassette, or a subject gene expression construct. Thus, in some embodiments, the present disclosure provides a genetically modified prokaryotic host cell, where the prokaryotic host cell is genetically modified with a nucleic acid comprising a subject synthetic translation regulator. In other embodiments, the present disclosure provides a genetically modified prokaryotic host cell, where the prokaryotic host cell is genetically modified with a nucleic acid comprising a subject gene expression cassette. In other embodiments, the present disclosure provides a genetically modified prokaryotic host cell, where the prokaryotic host cell is genetically modified with a subject gene expression construct.

To generate a subject genetically modified host cell, a subject nucleic acid (e.g., a nucleic acid comprising a subject synthetic translation regulator, a nucleic acid comprising a subject gene expression cassette, or a subject gene expression construct) is introduced stably or transiently into a parent host cell, using established techniques, including, but not limited to, electroporation, calcium phosphate precipitation, DEAE-dextran mediated transfection, liposome-mediated transfection, and the like.

Prokaryotic host cells include bacteria. The terms “bacteria” or “bacterium” include, but are not limited to, Gram positive and Gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasli, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof.

A subject genetically modified host cell can be used in research applications, e.g., to study regulation of a gene or genes. A subject genetically modified host cell can be used to produce a polypeptide encoded by a coding region of interest, e.g., to control production of the polypeptide.

### Methods of Modulating Gene Expression

The present disclosure provides methods of modulating gene expression (e.g., modulating translation of a coding region) in a bacterial cell. The methods generally involve contacting a bacterial cell with theophylline, where the cell is genetically modified with a nucleic acid comprising a subject synthetic translation regulator operably linked to a coding region. In the presence of theophylline, translation of the coding region is increased, compared to the level of translation of the coding region in the absence of theophylline.

A subject method can be carried out in a wide variety of bacterial cells, including, e.g., Gram positive and Gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasli, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. In some instances, the bacterium is a human pathogen.

In the presence of theophylline (e.g., from about 1 mM theophylline to about 2 mM theophylline), translation of the operably linked coding region is increased by from about 2-fold to about 100-fold, or more than 100-fold compared to the level of translation of the operably coding region in the absence of theophylline. For example, in the presence of theophylline, translation of the operably linked coding region is increased by from about 2-fold to about 5-fold, from about 5-fold to about 10-fold, from about 10-fold to about 25-fold, from about 25-fold to about 50-fold, or from about 50-fold to about 100-fold, or more than 100-fold, compared to the level of translation of the operably coding region in the absence of theophylline.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Synthetic Riboswitches that Induce Gene Expression in Diverse Bacterial Species

### Materials and Methods

Materials.

Synthetic oligonucleotides were purchased from Integrated DNA Technologies (Coralville, Iowa). Culture media was obtained from EMD Bioscience. Theophylline and o-nitrophenyl-β-D-galactopyranoside (ONPG) were purchased from Sigma. Kanamycin was purchased from Fisher Scientific. X-gal was purchased from US Biological. DNA polymerase and restriction enzymes were purchased from New England BioLabs. Plasmid manipulations were performed using E. coli MDS42 cells (Scarab Genomics) that were transformed by electroporation. Purifications of plasmid DNA, PCR products, and enzymatic digestions were performed by using kits from Qiagen. All new plasmids were verified by DNA sequencing performed by MWG Biotech or Elim Biopharmaceuticals.

Development of Riboswitch A.

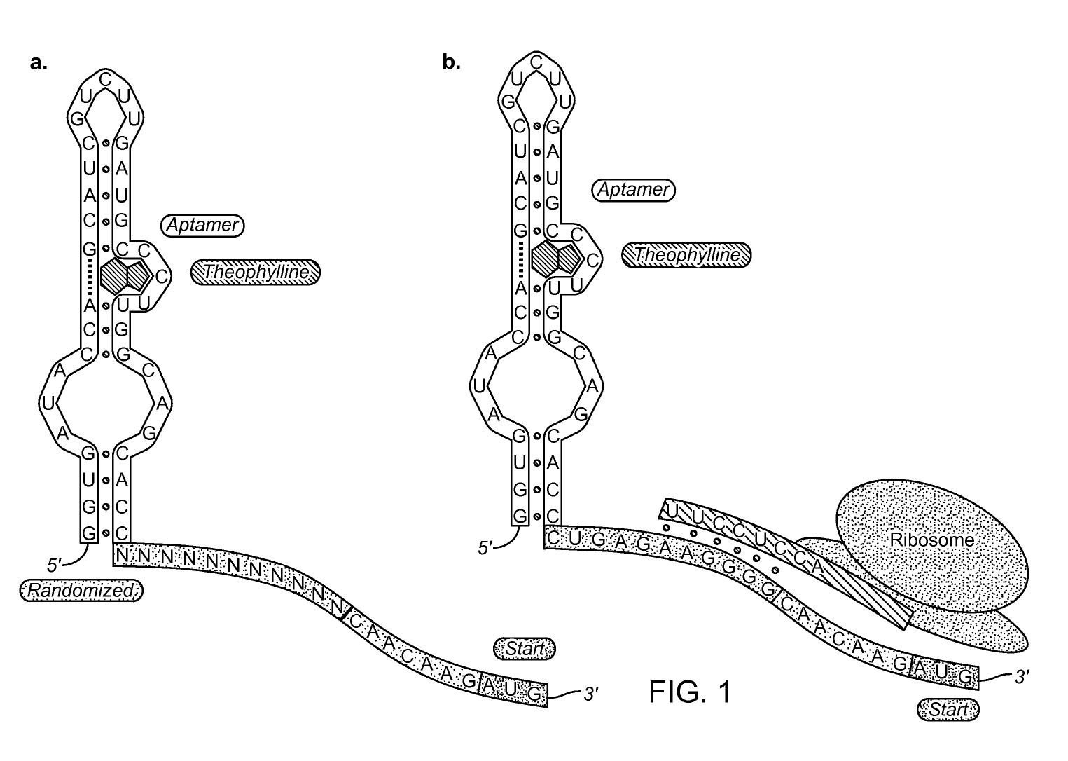

A previously reported high-throughput screen (9) was modified to isolate theophylline-sensitive riboswitches from the N11 library shown in FIG. 1A. The theophylline aptamer sequence is shown in green, the randomized region in blue, and the start codon in peach. Approximately 20,000 clones were screened directly in A. baylyi cells, and the sequence shown in FIG. 1B shows the predicted secondary structure of the mRNA for Riboswitch A in the ligand-bound ‘on’ state (18). Predicted pairing between the 16S rRNA and the putative RBS of the mRNA is shown.

Development of Riboswitch B.

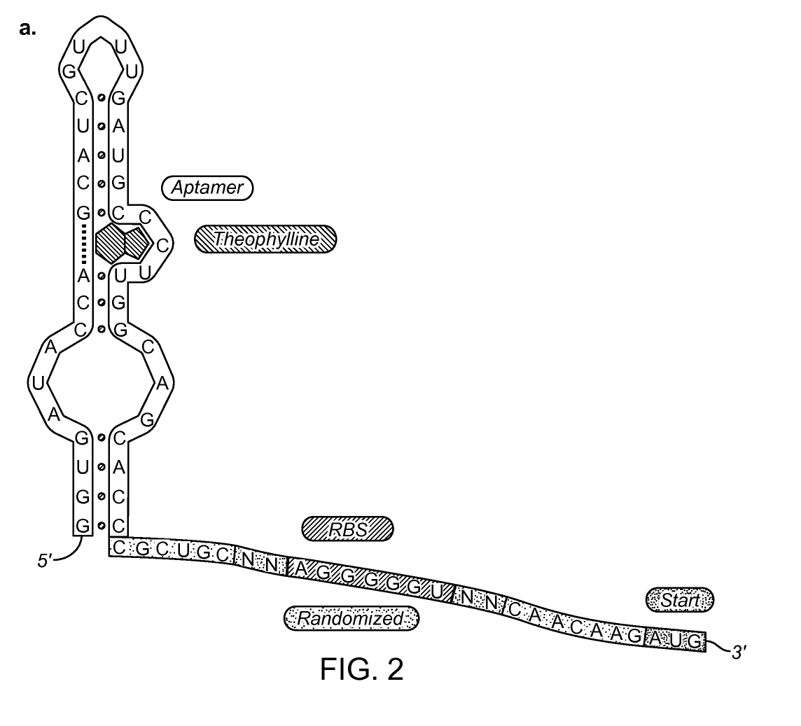

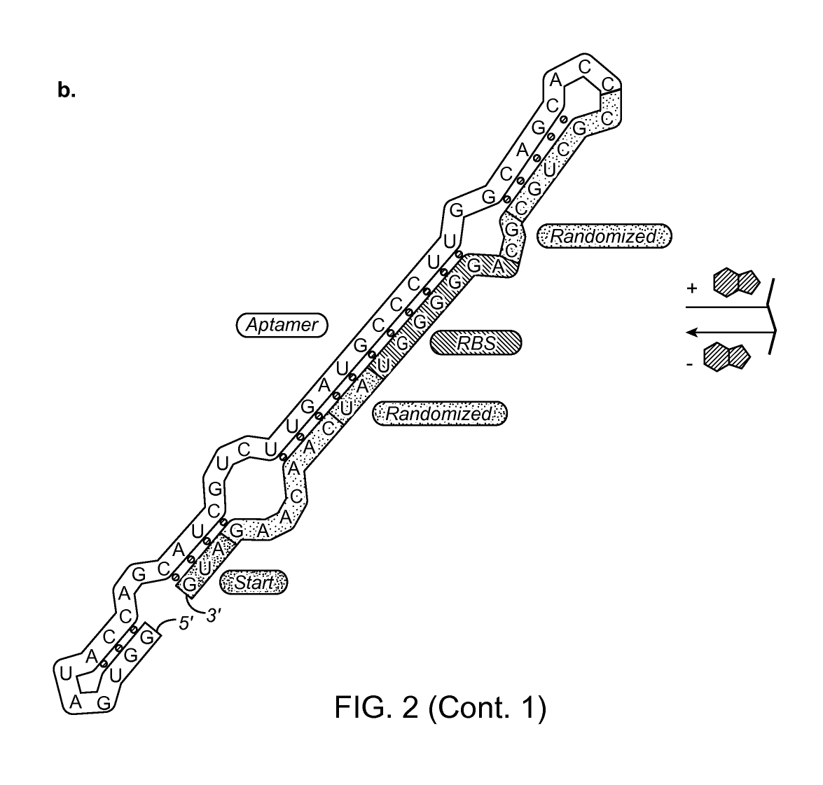

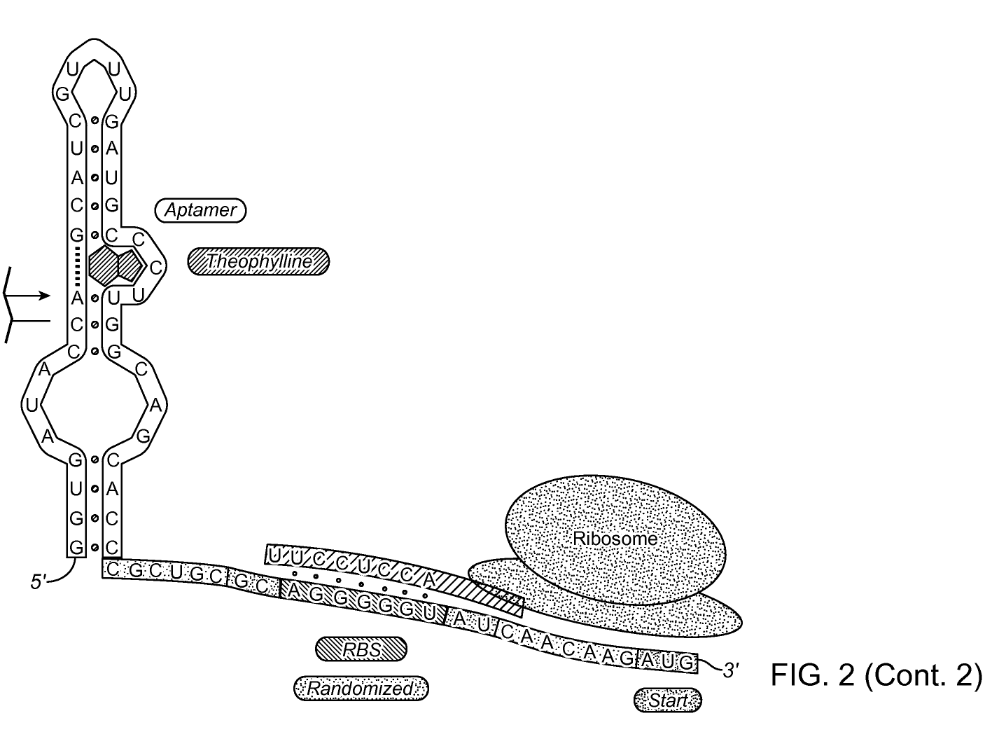

The library shown in FIG. 2A was designed by combining information from previously reported riboswitch selections (9, 10, 16) with in silico secondary structure predictions (18), to generate a 256 member library featuring a strong putative RBS and strong predicted pairing in the absence of ligand. The theophylline aptamer sequence is shown in green, the randomized region in blue, the putative RBS sequence in pink, and the start codon in peach. Assays were performed in E. coli and B. subtilis cells, and the sequence shown in FIG. 2B shows the predicted secondary structure of the mRNA for Riboswitch B in the ligand-free ‘off’ state (left) and the ligand-bound ‘on’ state (right). Predicted pairing between the 16S rRNA and the putative RBS of the mRNA is shown.

Development of Riboswitch C.

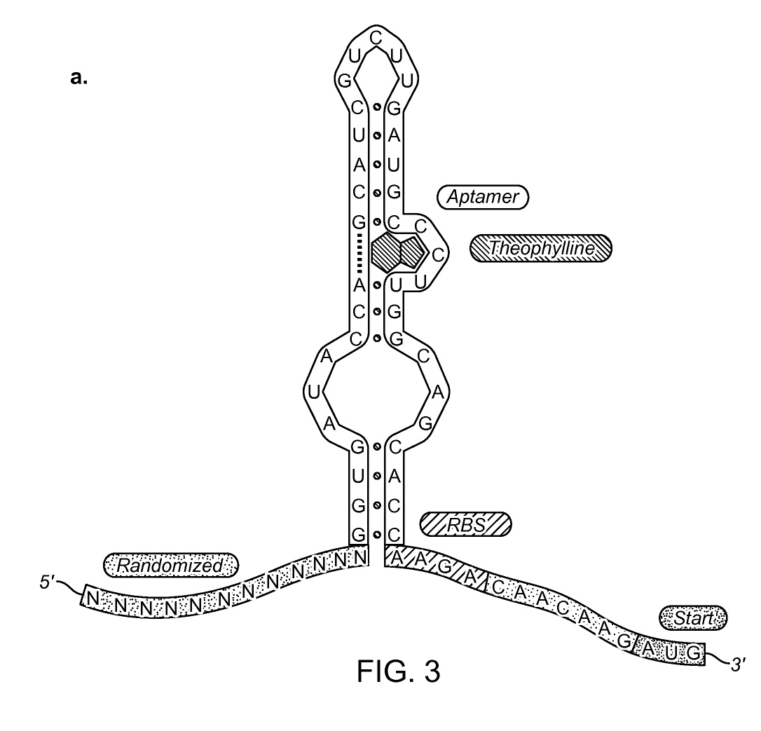

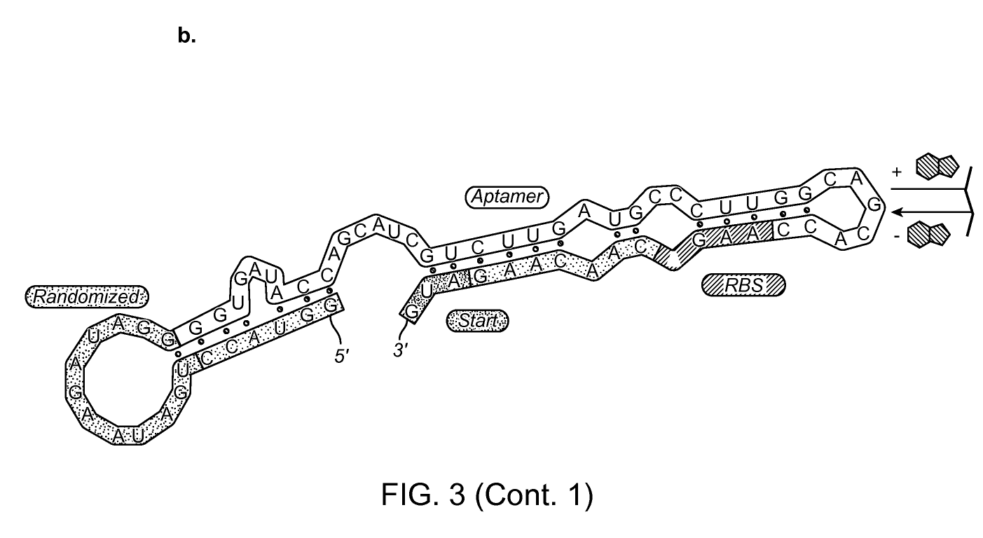

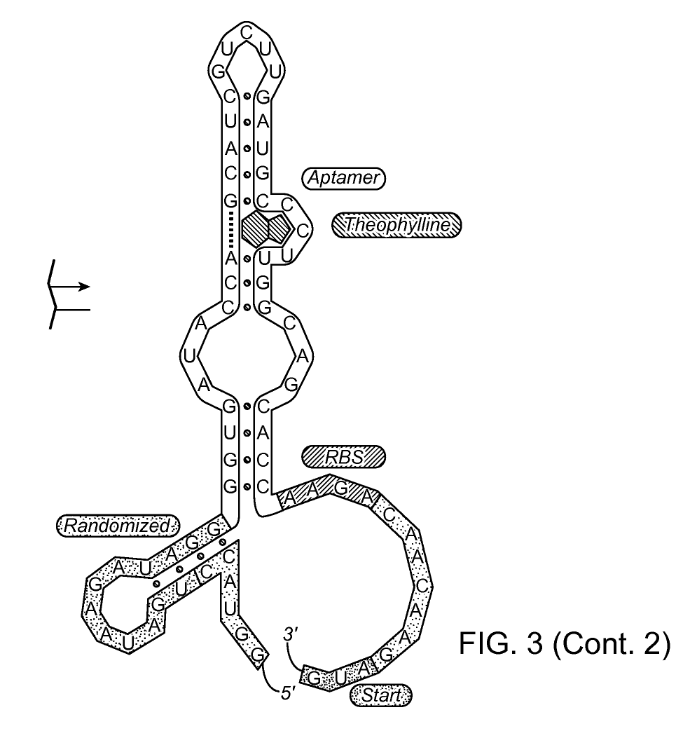

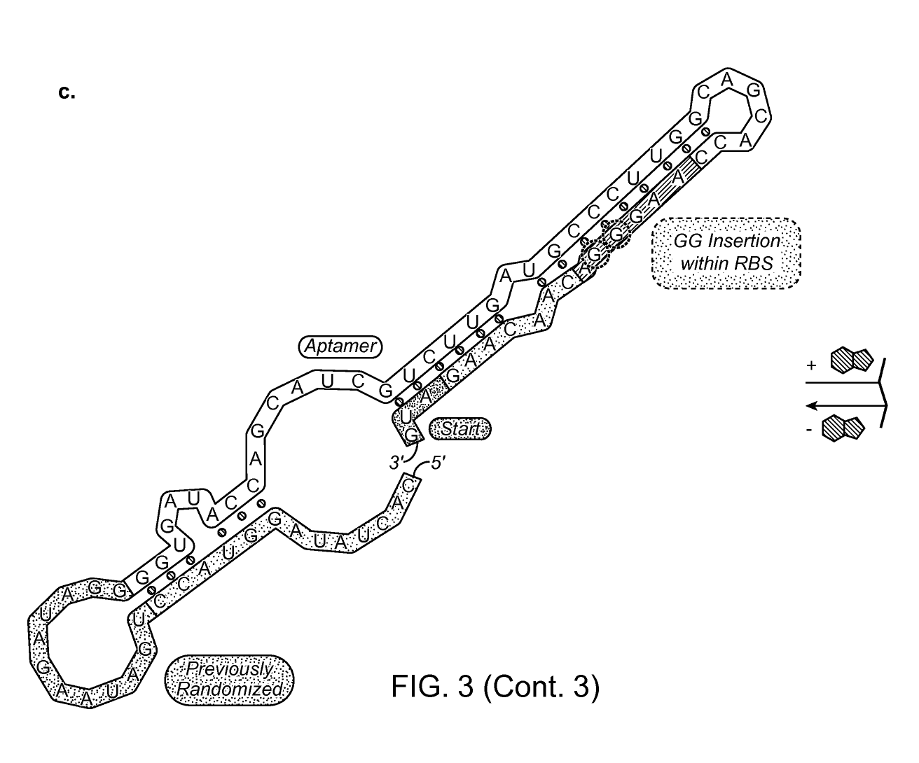

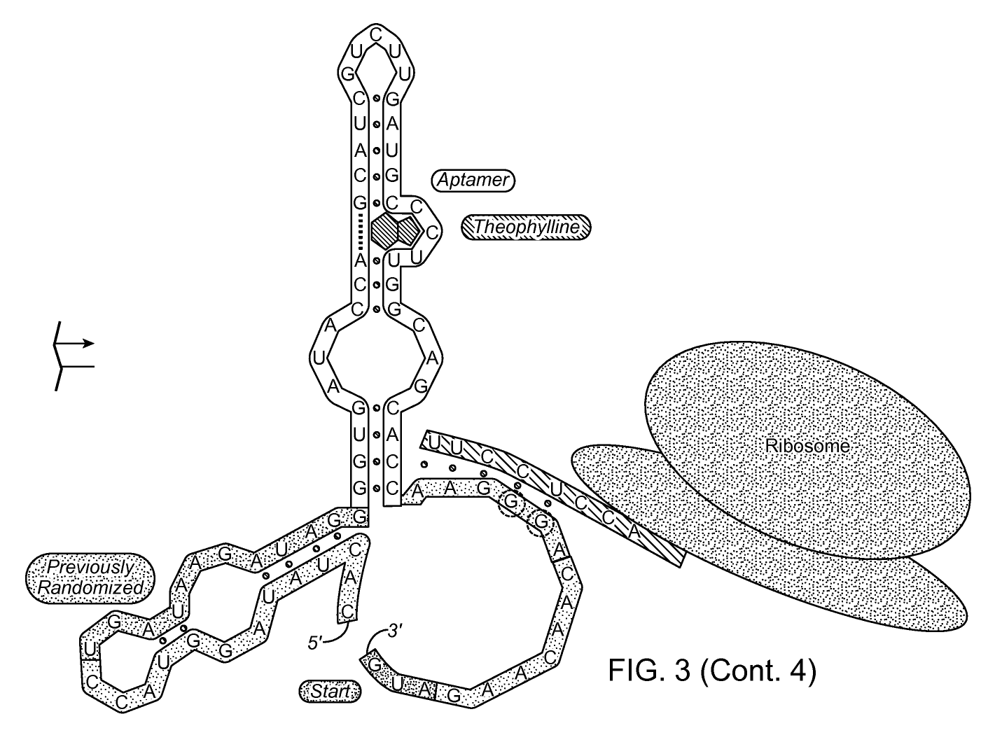

The library shown in FIG. 3A was designed by combining information from a previously reported riboswitch selection (9) with in silico secondary structure predictions (18), to generate a library in which a randomized region of 12 bases (blue) was positioned 5′ to the theophylline aptamer (green), and a relatively weak putative RBS (pink) was positioned 3′ to the aptamer stem. The start codon is shown in peach. High-throughput assays for riboswitch function were performed in E. coli cells, and FIG. 3B shows the predicted secondary structures in the ligand-free ‘off’ state (left) and the ligand-bound ‘on’ state (right) of a riboswitch that activates gene expression ˜10-fold in the presence of 1 mM theophylline. To obtain a riboswitch with a stronger RBS, we inserted ‘GG’ within the putative RBS sequence to obtain Riboswitch C. FIG. 3C shows the predicted secondary structure of the mRNA for Riboswitch C in the ligand-free ‘off’ state (left) and the ligand-bound ‘on’ state (right). Predicted pairing between the 16S rRNA and the putative RBS of the mRNA is shown.

Development of Riboswitches D and E.

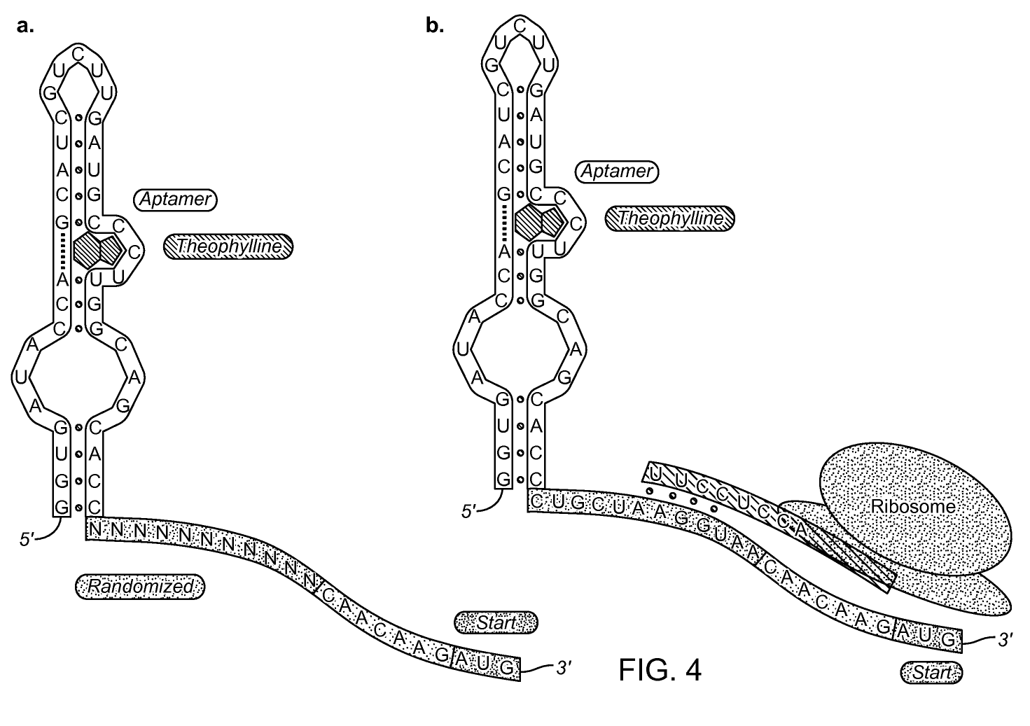

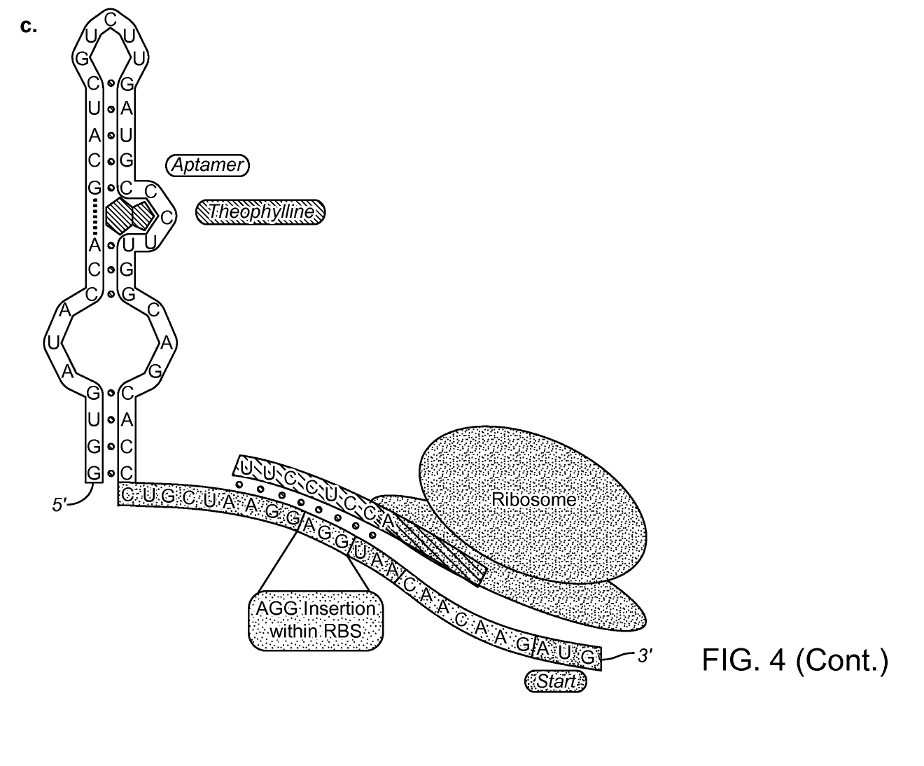

The library shown in FIG. 4A was screened as previously reported (10) to obtain the theophylline dependent Riboswitch D shown in FIG. 4B. Predicted pairing between the 16S rRNA and the putative RBS of the mRNA is shown. Rational design and in silico secondary structure predictions (18) were used to generate Riboswitch E, shown in (c.), by inserting the sequence ‘AGG’ (pink) within the RBS of its parent Riboswitch D. Riboswitch E has the consensus ‘AAGGAGG’ RBS sequence, and maintains similar predicted secondary structures as Riboswitch D. Predicted pairing between the 16S rRNA and the putative RBS of the mRNA is shown.

E. coli Manipulations.

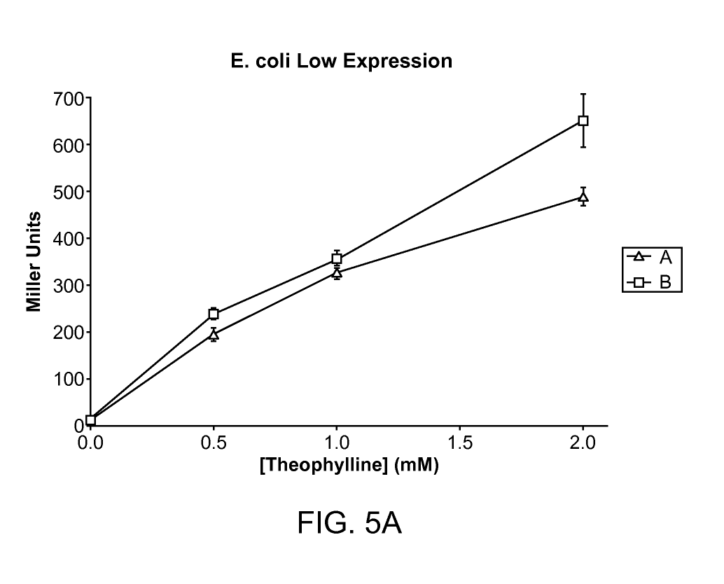

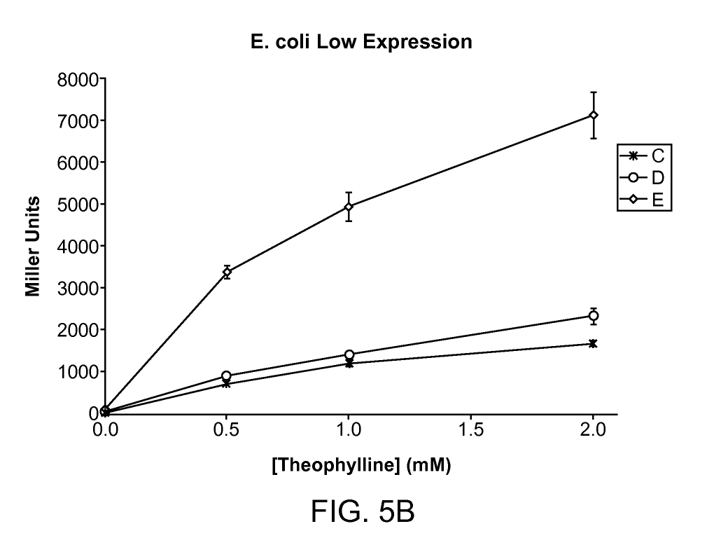

Electrocompetent E. coli cells (strain MDS42, Scarab Genomics) were prepared and transformed with the pBAV1K riboswitch constructs by electroporation. All β-galactosidase assays were performed by the method of Miller (13), using cells that were grown with shaking at 37° C., in Luria Broth (LB) containing kanamycin (50 μg/mL) and 0 or 2 mM theophylline. Dose response profiles in E. coli are shown in FIGS. 5A and 5B. Assays were performed in triplicate as described in the Methods section for E. coli. Error bars are ±SD.

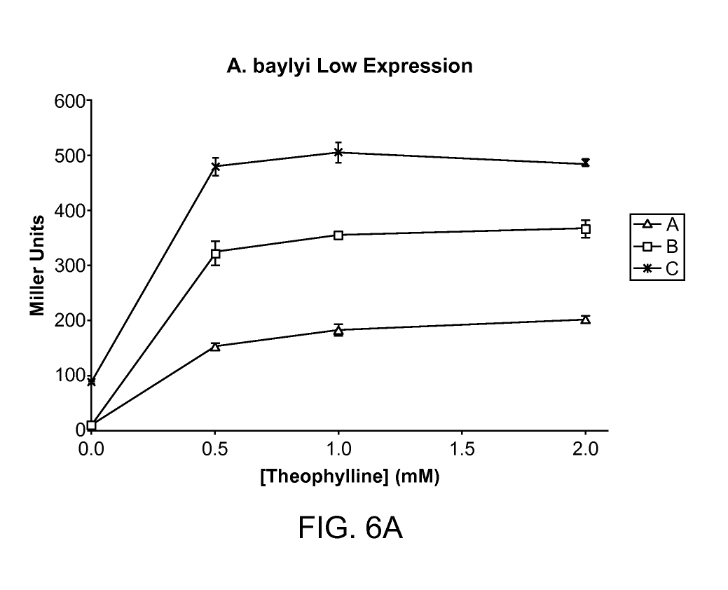

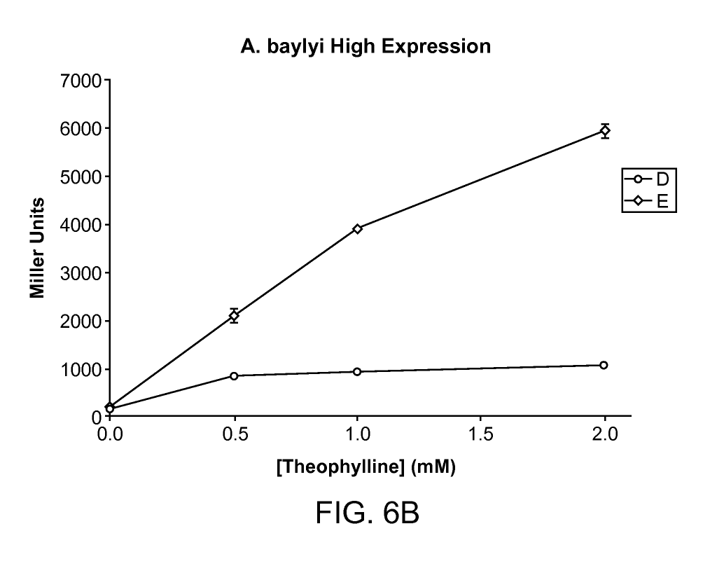

A. baylyi manipulations. To transform A. baylyi strain ADP1, ATCC 33305 (17) with the pBAV1K constructs, cells were grown overnight in 5 mL LB at 30° C. with shaking (250 rpm). The following morning, 3 mL fresh LB was inoculated with 200 μL from the overnight culture. These cells were grown at 30° C. for 90 min, at which time the culture was divided into 300 μL aliquots. 3 μL plasmid DNA was added to each tube of cells. These cultures were grown with shaking at 30° C. After 3 h, 200 μL of these cultures was plated on selective media. β-galactosidase assays were performed by the method of Miller (13), using cells that were grown with shaking at 37° C., in LB containing kanamycin (50 μg/mL) and 0 or 2 mM theophylline. Dose response profiles in A. baylyi are shown in FIGS. 6A and 6B. Assays were performed in triplicate as described in the Methods section for A. baylyi. Error bars are ±SD.

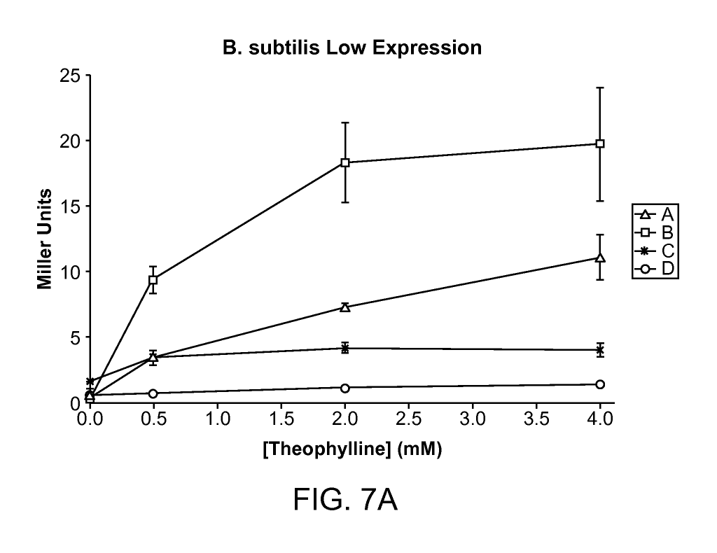

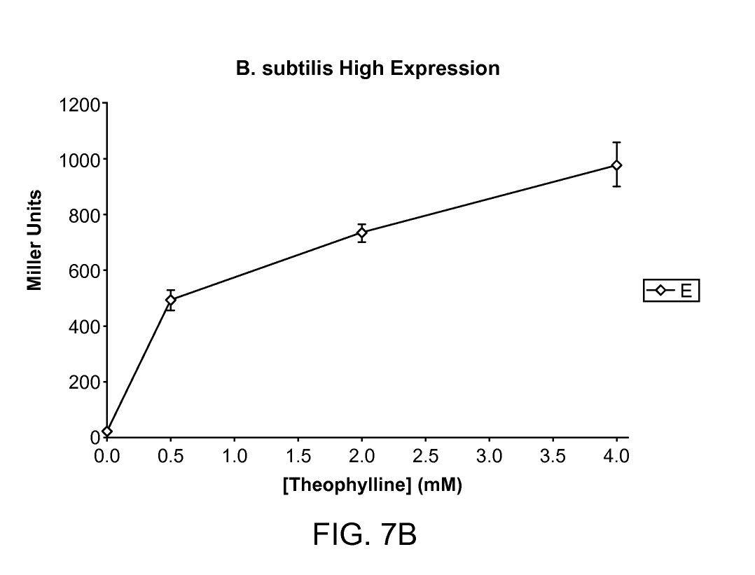

B. subtilis manipulations. B. subtilis strain JH642 (2) was grown at 37° C. in LB with shaking (250 rpm). Competent B. subtilis cells were prepared and transformed using the Spizizen method (5). Transformants were selected on LB with 20 μg/mL kanamycin. To assay for β-galactosidase activity, strains harboring the riboswitches were grown overnight in LB media with 20 μg/mL kanamycin. In the morning, cells were diluted 1:100 into fresh selective media containing 1 mM IPTG and either 0 or 2 mM theophylline. β-galactosidase assays were performed by the method of Miller (13) using permeabilization with toluene. Dose response profiles in B. subtilis are shown in FIGS. 7A and 7B. Assays were performed in triplicate as described in the Methods section for B. subtilis. Error bars are ±SD.

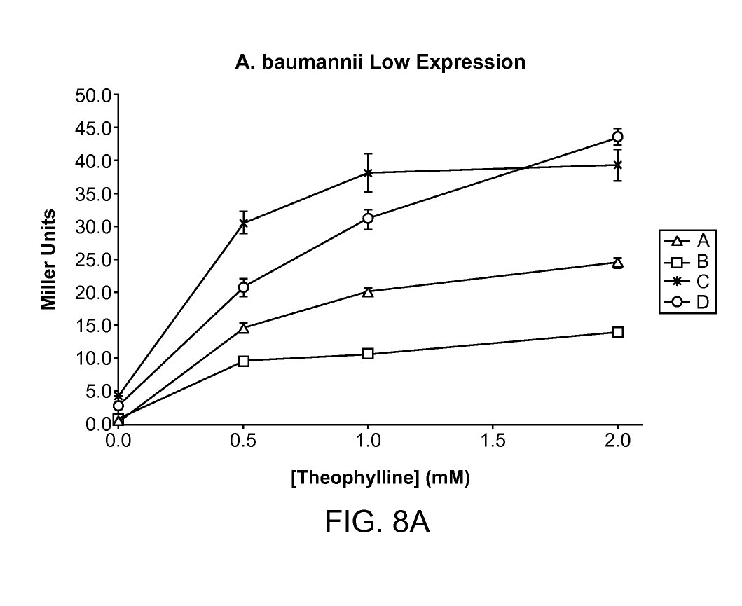

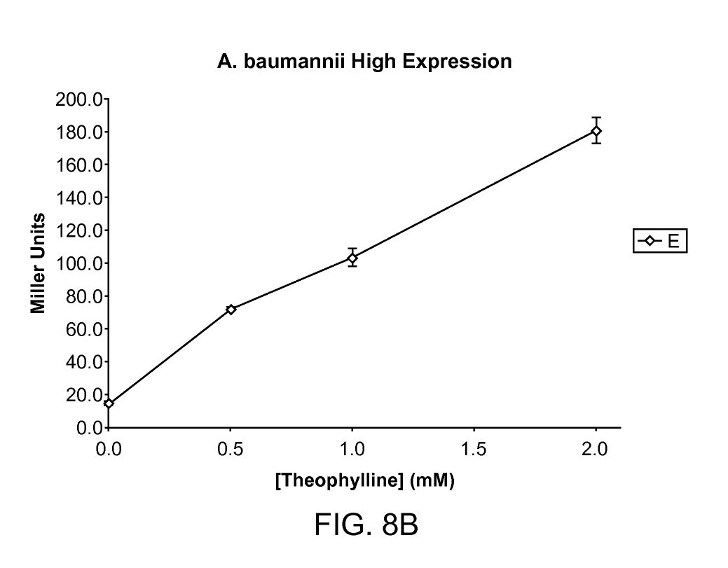

A. baumannii manipulations. Acinetobacter baumannii ATCC 19606 (1) was grown at 37° C. in LB with shaking. Electrocompetent cells were prepared by pelleting mid-log phase bacteria at 5000 g for 5 min, washing pellets twice with cold 2 mM CaCl₂, twice with 10% glycerol, and resuspending the cells in 10% glycerol. The electrocompetent A. baumanii cells were transformed using a BioRad electroporator in 0.1 cm cuvettes, setting 1.8 kV. The cells were recovered in LB at 37° C. and selected on LB plates with 30 μg/mL kanamycin. A. baumannii harboring pBAV1K plasmids were grown at 37° C. in LB-kanamycin (30 μg/mL) with shaking. A β-galactosidase assays were performed by the method of Miller (13), using cells that were grown with shaking at 37° C., in the presence of 0 or 2 mM theophylline. Dose response profiles in A. baumannii are shown in FIGS. 8A and 8B. Assays were performed in triplicate as described in the Methods section for A. baumannii. Error bars are ±SD.

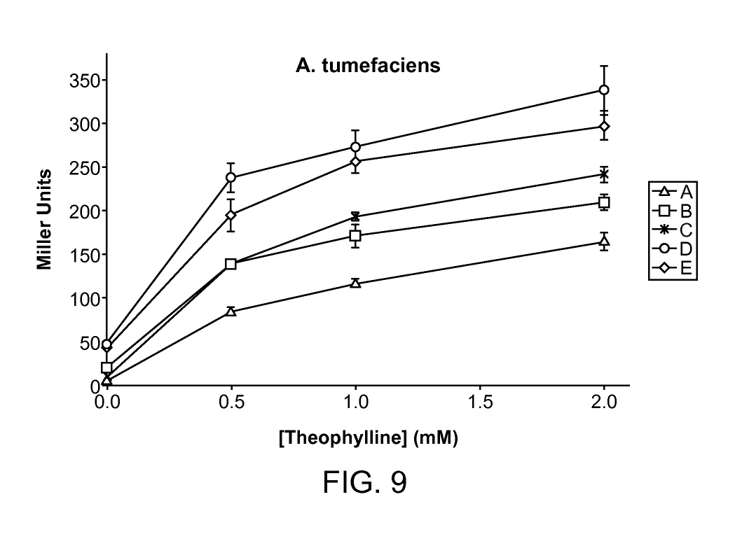

A. tumefaciens manipulations. Competent A. tumefaciens cells (strain C58 (7), gift from Dr. David Lynn, Emory University) were prepared and transformed by the method of Cangelosi et al. (3). Cells were grown in 5 mL LB at 28° C. with shaking (250 rpm). After 18 h, 10-100 μL of saturated culture was added to fresh LB (50-150 mL) and allowed to grow until the OD₆₀₀=0.4. The cells were then pelleted three times by centrifugation at 5000 g, washing twice with water and once with 10% glycerol. 1 μL of plasmid DNA was mixed with 50 μL of cells, which were transformed by electroporation at 1800 V (Eppendorf Electroporater 2510; Westbury, N.Y.). Electroporated cells were permitted to recover in 500 μL SOB for 4 h 28° C. Finally, 10-100 μL of culture was plated on selective media. All β-galactosidase assays were performed by the method of Miller (13), using cells that were grown with shaking at 28° C., in the presence of 0 or 2 mM theophylline. A dose response profile in A. tumefaciens is shown in FIG. 9. Assays were performed in triplicate as described in the Methods section for A. tumefaciens. Error bars are ±SD.

M. smegmatis Manipulations.

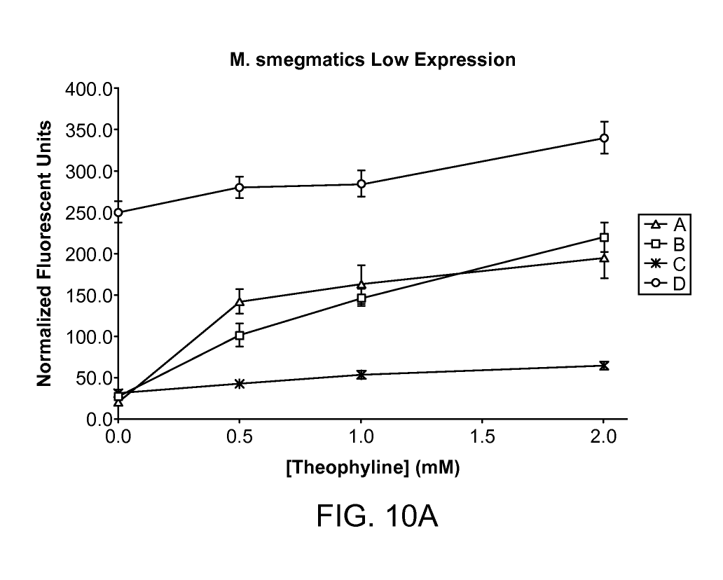

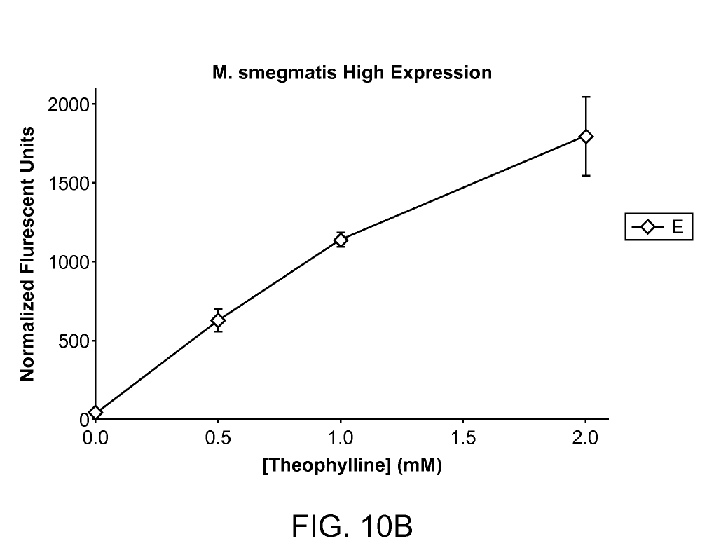

M. smegmatis mc²155 (15) was grown at 37° C. in 7H9 liquid media or on 7H11 agar (Difco) containing 0.5% glycerol, 0.5% glucose, 0.05% Tween 80 and 20 μg/mL kanamycin unless otherwise noted. Plasmids were electroporated into M. smegmatis and plated on selective media for 3 days. Transformants were grown overnight in 7H9 to an optical cell density in 1 cm at 600 nm (OD₆₀₀) of 1-2. Cells were exchanged into fresh 7H9 containing 0 or 2 mM theophylline to OD₆₀₀ 0.3 in 1 mL per media condition in 24-well plates and incubated for 6 hrs (2 doubling times) at 215 rpm. The OD₆₀₀ was measured prior to resuspending the pelleted cells in 200 μL PBS containing 0.05% Tween 80. Fluorescence was measured in a plate reader at 510 nm with 450 nm excitation and 495 nm cutoff (SpectraMax Gemini XPS, Molecular Devices) and normalized to OD₆₀₀. Cells transformed with the plasmid pMV261 were used as control for background fluorescence. Each growth condition was performed in triplicate. Dose response profiles in M. smegmatis are shown in FIGS. 10A and 10B. Assays were performed in triplicate as described in the Methods section for M. smegmatis. Error bars are ±SD.

S. pyogenes Manipulations.

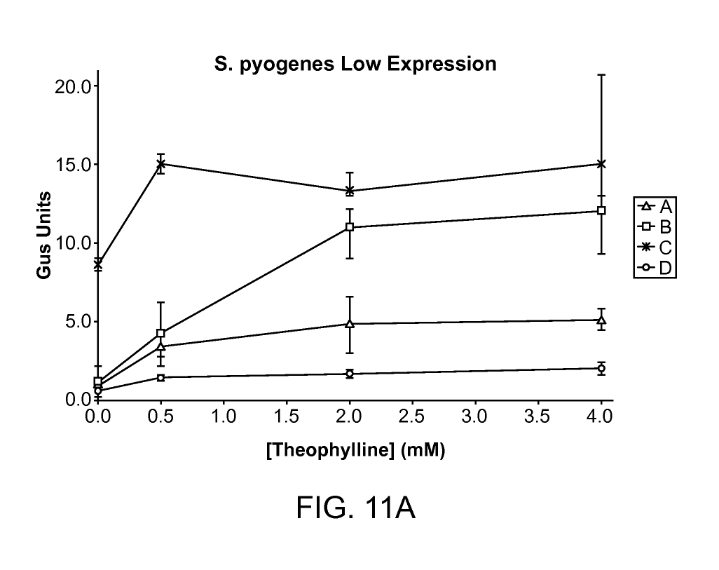

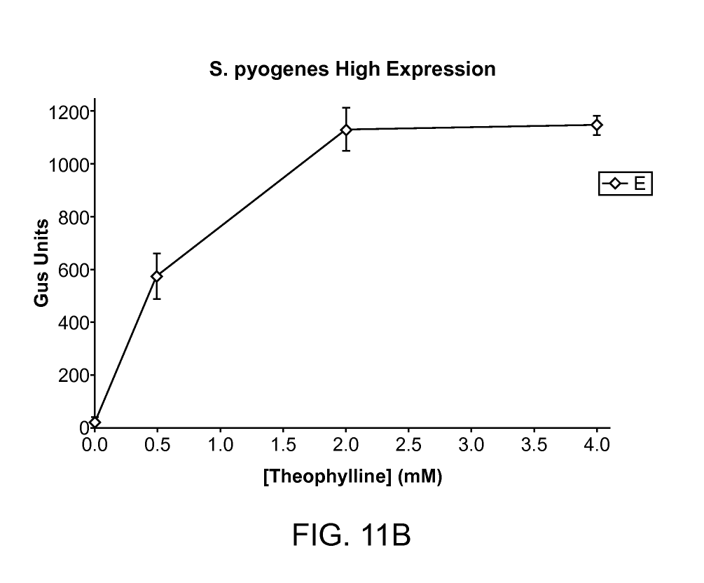

GAS strain JRS1278 (T. C. Barnett and J. R. Scott, unpublished data) was transformed with the riboswitch plasmids using a previously reported method (4). JRS1278 is ΔcovR::cat in MGAS315 generated using pJRS1349, as previously described (6). To assay for β-glucuronidase activity, riboswitch-harboring strains were grown overnight in Todd-Hewitt yeast extract broth (THY broth) containing 100 μg/mL spectinomycin. This overnight culture was then divided into 1.25 mL aliquots into conical flasks containing 23.75 mL pre-warmed THY broth containing 0 or 2 mM theophylline. These dilutions were divided equally into three 15-mL conical flasks (8 mL each), and were grown in a 37° C. water bath without agitation until 2 hours into stationary phase, as determined by following the optical density. Cultures were chilled on ice for 10 min and were then pelleted at 3250 g for 10 min at 4° C. The supernatant was discarded and the pellets were resuspended in remaining supernatant and transferred to 1.5 mL Eppendorf tubes. The resuspended cells were pelleted, the supernatant was removed, and the dry pellet was stored at 4° C. For the assay, cell pellets were resuspended in 1 mL of ice-cold Z buffer and lysed in tubes with a glass bead matrix via vortexing at maximum speed for 30 minutes. Cell debris was pelleted and cell lysates were transferred to fresh 1.5 mL tubes. Lysates were assayed by addition of 4 mg/ml solution of p-nitrophenyl β-D-glucuronide in Z buffer and the OD₄₂₀ kinetic curve was recorded. Protein concentrations of lysates were determined using a BCA protein assay kit (Thermo). Gus activity was determined by dividing the OD₄₂₀ by protein concentration (in μg/mL), multiplying by 1000 and dividing by the time in minutes. Dose response profiles in S. pyogenes are shown in FIGS. 11A and 11B. Assays were performed in triplicate as described in the Methods section for S. pyogenes. Error bars are +SD.

M. magneticum Manipulations.

All riboswitches for M. magneticum were cloned within the 5′-UTR of pAK22 (8), which features the Ptac promoter and expresses a MamK-green fluorescent protein (GFP) translational fusion. Cells of M. magneticum strain AMB-1 (12) were transformed with the constructs A-E by conjugation as previously described (14). The strains were grown at 30° C. in MG growth media in the presence of kanamycin (10 μg/mL) in a chamber with the oxygen concentration maintained below 10%. Starting from a culture that had been grown 24 h and had reached exponential phase (OD₄₀₀=0.1), two 10 mL sub-cultures were inoculated at an initial OD₄₀₀ of 0.05, in MG media in the absence or presence of 1 mM theophylline. The cells were grown in micro-aerophilic conditions and 100 μL of cells were spun down at different time points after inoculation (50 minutes to 24 hours), spotted on agarose pads prepared with 1% agarose in growth media, and imaged under phase contrast and fluorescence microscopy as described (14). All fluorescent images were exposed for six seconds and the cells were visualized with the 100× objective.

Adaptation of Riboswitch Constructs for Use in New Bacterial Species.

It is anticipated that in many bacterial species, Riboswitches A-E may be characterized in the context of the broad-host range vector, pBAV1K, using the T5 promoter and the lacZ reporter gene. This set of riboswitches is available upon request, and has also been contributed to the American Type Culture Collection (ATCC). (Five of the eight species described in this study were transformed and assayed for β-galactosidase activity using these unmodified plasmids.) However, a utility of these genetic control elements is that they function not only in a broad range of bacterial species, but that they can also be used in concert with a variety of plasmids, promoters, and reporter genes. Here, technical considerations are described for constructing Riboswitches A-E in the context of a different plasmid, promoter, or reporter gene. These riboswitches may also be inserted at a chromosomal locus of interest.

Riboswitches A-E were cloned into the broad host range vector pBAV1K to enable modular subcloning of various promoter, riboswitch, and reporter gene sequences. The plasmid map shown at right, and the diagram shown below, highlight several unique restriction sites that may be useful to modify various features. The promoter and constant 5′-UTR sequence (described in the text of Table 1) is positioned between XbaI and KpnI sites; the riboswitch sequence (Table 1) is positioned between the KpnI site and the start codon; and the stop codon of the reporter gene (IS10-lacZ, in this case) precedes the SpeI recognition site. An intrinsic transcriptional termination sequence is positioned 3′ to the ApaI restriction site, and pBAV1K also features transcriptional terminators in either direction flanking the multiple cloning sites to prevent undesired transcription from promoters elsewhere on the plasmid through the mRNA of interest.

TABLE 1
Sequences of Riboswitches A-E.

| | Riboswitch Sequence |
|---|---|
| A | GGUACCGGUGAUACCAGCAUCGUCUUGAUGCCCUUGGCAGCACCCUGAGAAGGGGCAACAAGAUG |
| | (SEQ ID NO: 11) |
| B | GGUACCGGUGAUACCAGCAUCGUCUUGAUGCCCUUGGCAGCACCCGCUGCGCAGGGGGUAUCAACAAGAUG |
| | (SEQ ID NO: 12) |
| C | GGUACCUGAUAAGAUAGGGGUGAUACCAGCAUCGUCUUGAUGCCCUUGGCAGCACCAAGGGACAACAAGAUG |
| | (SEQ ID NO: 13) |
| D | GGUACCGGUGAUACCAGCAUCGUCUUGAUGCCCUUGGCAGCACCCUGCUAAGGUAACAACAAGAUG |
| | (SEQ ID NO: 14) |
| E | GGUACCGGUGAUACCAGCAUCGUCUUGAUGCCCUUGGCAGCACCCUGCUAAGGAGGUAACAACAAGAUG |
| | (SEQ ID NO: 15) |
| E* | GGUACCGGUGAUACCAGCAUCGUCUUGAUGCCCUUGGCAGCACCCUGCUAAGGAGGCAACAAGAUG |
| | (SEQ ID NO: 16) |

The mRNA sequence of each riboswitch is shown, with the KpnI site italicized, aptamer sequence underlined, RBS sequences bold and underlined, and start codon bolded. The transcripts also had a constant region 5′ to the sequences shown (5'- . . . AUACGACUCACUAUA (SEQ ID NO: 19), which was preceded by an additional untranslated sequence that was constant for all switches tested within a given organism, but varied across the species tested. Riboswitch E* was derived from Riboswitch D, by replacing ‘UAA’ with ‘AGG’ to obtain the consensus prokaryotic RBS sequence. Riboswitch E* is an alternative to Riboswitch E for use in Gram-positive bacteria that typically have shorter spacing between the RBS and start codon, such as Mycobacterium tuberculosis (11). In most species tested, Riboswitch E* has lower background gene expression but also a smaller dynamic range than Riboswitch E.

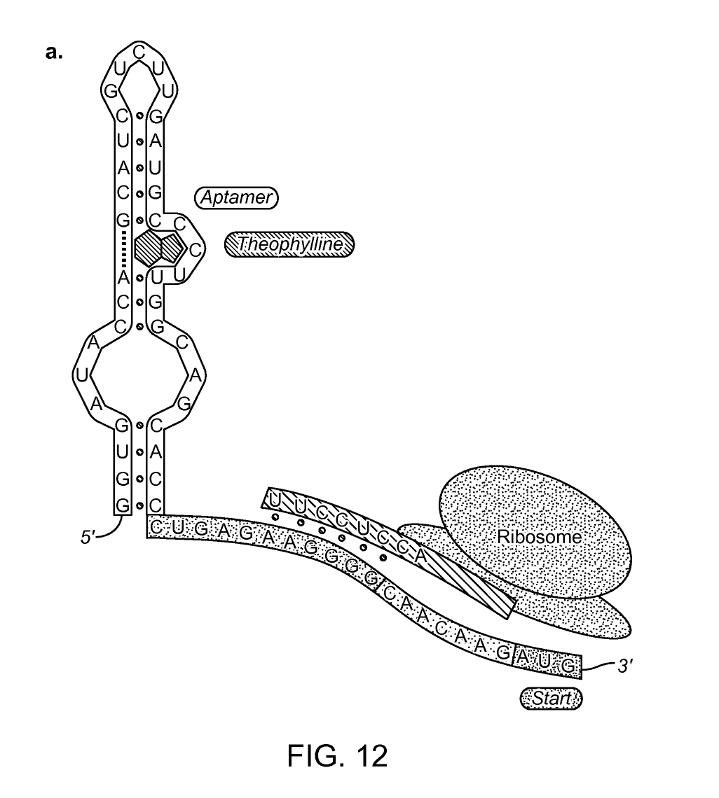

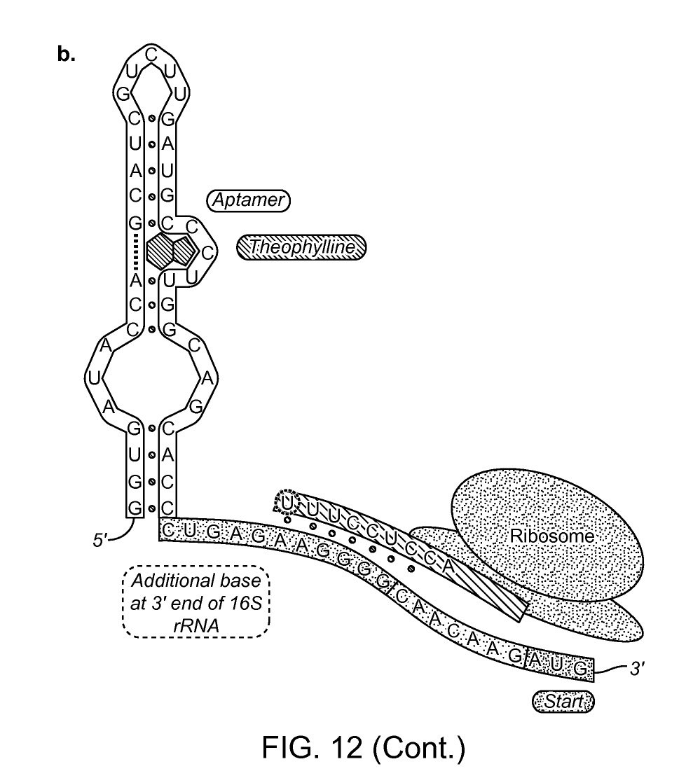

FIGS. 12A and 12B—Comparison of A. tumefaciens or M. magneticum 16S rRNA pairing with the RBS of Riboswitch A.

The 16S rRNA of A. tumefaciens strain C58 (FIG. 12A) is predicted to form 6 base pairs with the RBS of Riboswitch A, while the 16S rRNA of M. magneticum strain AMB 1 (FIG. 12B) is predicted to form 7 base pairs with the RBS of Riboswitch A. It was hypothesized that this additional base pairing interaction may increase both the background gene expression and the induced gene expression of Riboswitch A in M. magneticum compared to A. tumefaciens. This prediction was consistent with the observed results.

Some possible parameters for changing the plasmid, promoter, or gene of interest to test Riboswitches A-E in new bacterial species are provided below.

Plasmid—Riboswitches can be inserted at a chromosomal locus or can be cloned onto any plasmid that can be replicated and maintained in the species of interest. Transcriptional terminators can be placed 5′ and 3′ to the expression cassette, to prevent transcriptional read-through initiated by promoters elsewhere on the plasmid or chromosome.

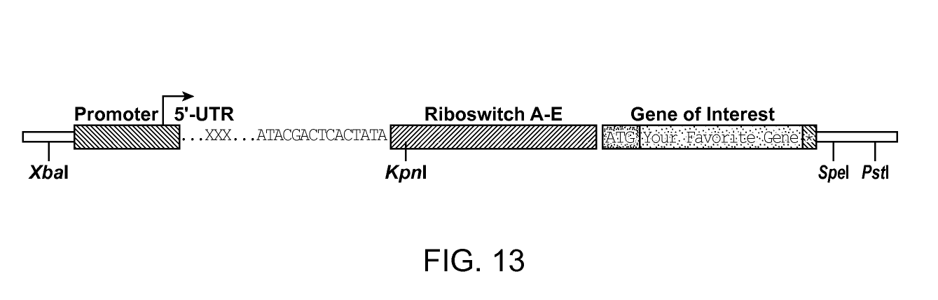

Promoter—The riboswitches described here act at the translational, not the transcriptional level. Thus, the choice of promoter is flexible. In cases where the promoter has been well characterized, long leader sequences and regulatory elements that are not essential for a given study can be removed. The first 15-30 bases that are transcribed by the native promoter can be positioned before the constant sequence ATACGACTCACTATA (SEQ ID NO:10), as indicated in the ???figure above??? and in Table 1. This approach was applied to construct the plasmids reported in this study for use in Mycobacteria smegmatis. In cases where the promoter sequence is poorly characterized, contains important regulatory elements, or requires a long leader sequence, the entire native 5′-UTR up until the native RBS, which must be removed to enable the riboswitches to function properly, can be used. The constant sequence ATACGACTCACTATA (SEQ ID NO:10), riboswitch sequence, and start codon can then be positioned as indicated in FIG. 13 and in Table 1. This approach was applied to construct the plasmids reported in this study for use in Streptococcus pyogenes.

Gene of Interest—Because the regulatory elements are located entirely in the 5′-UTR, these can likely be used to regulate the expression of most genes. The 4 examples reported here include: lacZ, gus, GFP, and mamK-GFP). While it is formally possible that certain sequences in the coding region may interfere with the riboswitch; such sequences can be modified using silent mutations. It is important to position the translational start codon as shown in Table 1, and to use the indicated RBS and flanking sequences.

### Results

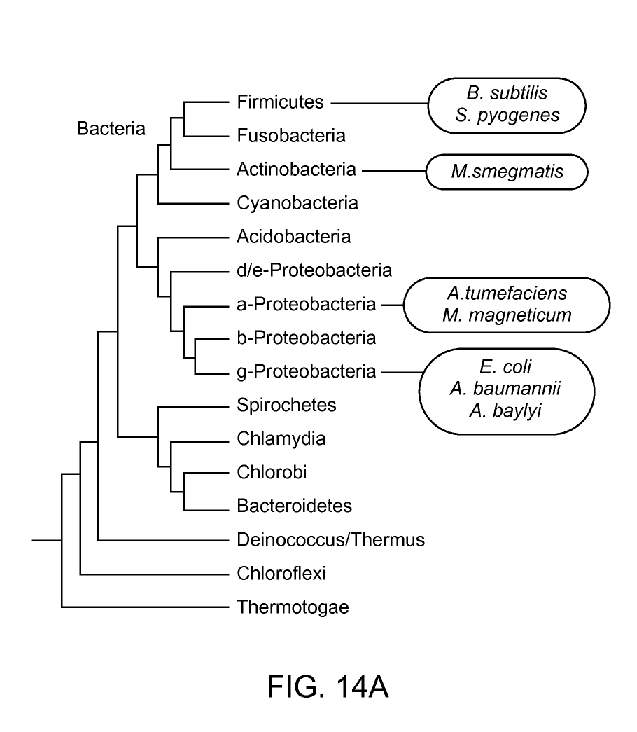

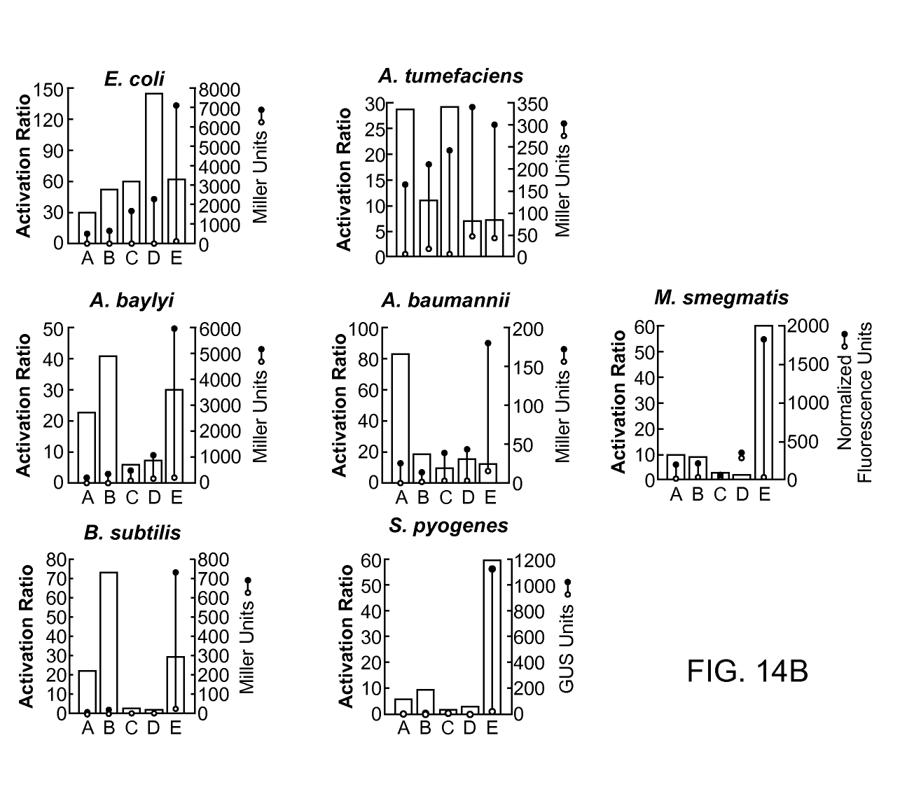

Using a combination of rational design and in vivo screening (see FIGS. 1-4), a set of five synthetic riboswitches was developed that enable inducible gene expression in eight diverse bacterial species (FIG. 14A), including organisms that currently have few or no tools to titrate gene expression in the laboratory. Three of the organisms are Gram-negative γ-proteobacteria, including E. coli; Acinetobacter baylyi strain ADP1 (22, 26), which is naturally competent but has few methods available for the conditional control of gene expression; and Acinetobacter baumannii (19), which is an opportunistic human pathogen that is often multi-drug resistant and can cause severe pneumonia in immuno-compromised patients, and currently appears to lack laboratory-inducible genetic control elements. Additionally, the switches were tested in the Gram-negative α-proteobacterium Agrobacterium tumefaciens (25), which is the causative agent of crown gall disease in dicot plants, and is widely used in genetic engineering applications (5). The switches were also in three Gram-positive bacteria, including the firmicutes Bacillus subtilis and Streptococcus pyogenes. B. subtilis is a well-studied model organism, while S. pyogenes is a human pathogen that causes several diseases including pharyngitis (“strep throat”), cellulitis, scarlet fever, and necrotizing fasciitis (4). Development of inducible control elements for S. pyogenes is particularly desirable because the only available expression system typically requires two separate plasmids, and is based on nisin, which is itself an antimicrobial agent (8). Finally, the riboswitches were tested in the actinobacterium Mycobacterium smegmatis, which is closely related to the pathogenic M. tuberculosis.

To construct the series of riboswitches in a broad-host range vector, we subcloned each riboswitch sequence (See Table 1 and FIG. 1-4) into pBAV1K, which features a T5 promoter and a lac operator sequence. Each pBAV1K-derived plasmid expresses the lacZ reporter gene. To construct the series of riboswitches for M. smegmatis, each sequence was cloned within the 5′-UTR of the eGFP gene of pMWS114. The vector pMWS114 contains the enhanced green fluorescent protein (eGFP) gene (S65T/F64L) cloned as a EcoRI-HindIII fragment into pMV261 (20). To construct riboswitch plasmids for S. pyogenes each sequence was introduced by inverse polymerase chain reaction (PCR) on the template pEU7742, which features the Psag promoter and the gusA reporter gene. A guide for adapting the riboswitch constructs for use in new bacterial species is provided below.

In every organism tested (see Materials and Methods; and FIGS. 5-11), at least one of the 5 riboswitches provided low levels of background expression in the absence of the ligand, and at least a 25-fold increase in gene expression in the presence of 2 mM theophylline (FIG. 14B). Activation ratios greater than 50-fold were achieved in most organisms, but it is important to note that switches that achieve the highest activation ratios often do so by having the lowest background expression in the absence of ligand. For applications that demand high levels of gene expression, a switch with a larger signal (but lower activation ratio) may be desirable. In nearly all organisms studied, there are at least two switches that display comparable activation ratios, but substantially different levels of expression (e.g., switches B and E in B. subtilis). In general, both background and signal increase moving from switches A to E, which is consistent with the presence of stronger ribosome binding sites or less stable secondary structures in the ligand-free states in switches C-E; full details of the RBS sequences are shown in Table 1. Because expression is dose-dependent (see FIGS. 5-11), it should be possible to achieve the desired expression level in a given application by titrating the concentration of theophylline.

With synthetic riboswitches validated in 7 organisms across 4 bacterial phyla, the ability of these switches to control gene expression in an organism not in the initial test set was assessed. In addition to demonstrating the utility of these switches to address a previously difficult genetic study, these experiments will determine whether the results in FIG. 14B have predictive value for choosing which switches are likely to perform well in a different organism. Magnetospirillum magneticum strain AMB-1 (16) is an aquatic α-proteobacterium that is able to navigate along Earth's magnetic field lines using a magnetite-containing membrane-bound organelle called the magnetosome. The study of magnetotactic bacteria is providing new insights into the process of biomineralization as well as a better understanding of organelle evolution and biogenesis in prokaryotes. The magnetotactic response of AMB-1 is dependent on the chain organization of the magnetosomes in the cell body, which requires expression of the actin-like cytoskeletal protein, MamK (12). Previous work has shown that the chain organization defect observed in a mamK deletion mutant can be restored by the constitutive expression of a GFP-tagged version of MamK from a plasmid (12). The tunable expression system described here will allow more precise control over MamK levels thus enabling time-resolved induction or depletion studies.

FIGS. 14A and 14.

Bacterial phylogeny. Species studied here are shown in the ovals. B) Activation ratios and expression levels for riboswitches A-E in each organism. Right axes: Expression levels in the absence of theophylline (open circles) and in the presence of theophylline (2 mM, closed circles). Measurements are of β-galactosidase activity in Miller units (17) for all organisms, except for S. pyogenes (β-glucuronidase activity in GUS units (11)) and M. smegmatis (normalized fluorescence of GFP (6)). Errors are smaller than the symbol size. Left axes: Activation ratios of the riboswitches, which are determined by dividing the expression level in the presence and absence of theophylline.

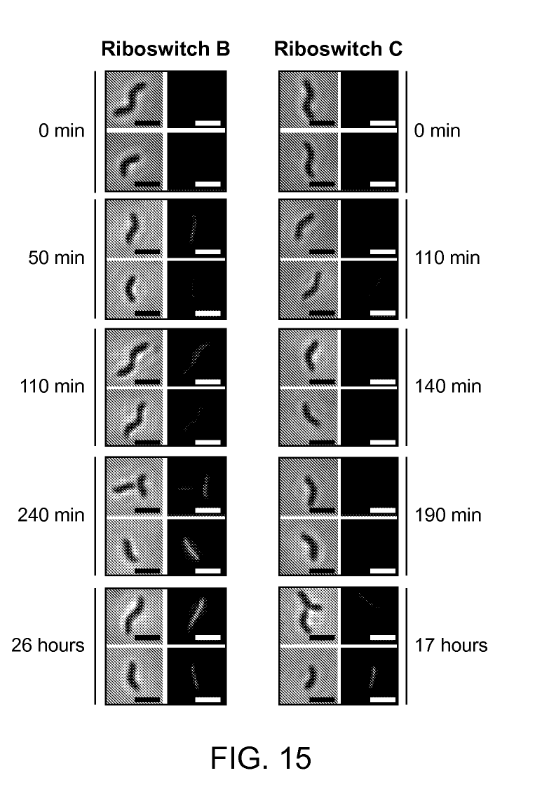

Based on data obtained in A. tumefaciens, which is also an α-proteobacterium and is cultured at a similar temperature (28° C. vs. 30° C. for M. magneticum), it was predicted that switches A, B, or C would be best suited for inducing actin-like filament formation in M. magneticum, while switches D and E would likely exhibit high levels of background gene expression in the absence of theophylline. However, consideration of the 16S rRNA sequences of each species suggested that switch A may exhibit higher background and induced gene expression in M. magneticum than in A. tumefaciens due to an additional possible base pairing interaction between M. magneticum 16S rRNA and the ribosome binding site of switch A (see FIG. 15). To test these hypotheses, each riboswitch was subcloned into the previously reported mamK-GFP expression plasmid (12) and imaged cells harboring these constructs at several time points following induction with 1 mM theophylline (see Materials and Methods). While all five riboswitches showed increases in MamK-GFP expression in the presence of theophylline, switches A, D, and E displayed detectable levels of MamK-GFP expression in the absence of the inducer. Consistent with our predictions, riboswitches B and C produced no visible expression of MamK-GFP in the absence of the inducer. At comparable times post induction with theophylline (1 mM), riboswitch B produced higher levels of MamK-GFP than riboswitch C, as shown by the appearance of fluorescent filaments extending from pole to pole (FIG. 15), but both riboswitches produced levels of MamK-GFP suitable for magnetosome localization studies (12, 18).

In summary, a series of synthetic riboswitches have been developed that function as genetic control elements in a diverse set of Gram-negative and Gram-positive bacteria. Using theophylline, an inexpensive small molecule that is non-toxic at the concentrations used here, at least 25-fold increase in gene expression in all species tested, and greater than 50-fold induction in two human pathogens, was observed.

FIG. 15. Images of riboswitches B and C controlling the expression of a MamK-GFP fusion in M. magneticum in the presence of theophylline (1 mM) as a function of time. Left panels are phase-contrast images, the right panels monitor GFP fluorescence emission (2 μm scale bar). All fluorescent images were exposed for six seconds and the cells were visualized with the 100× objective.

### REFERENCES

1. Bouvet, P. J. M., and P. A. D. Grimont. 1986. Taxonomy of the genus Acinetobacter with the recognition of Acinetobacter baumannii sp. nov., Acinetobacter haemolyticus sp. nov., Acinetobacter johnsonii sp. nov., and Acinetobacter junii sp. nov. and emended descriptions of Acinetobacter calcoaceticus and Acinetobacter lwoffii. Int. J. Syst. Bacteriol. 36:228-240.
2. Brehm, S. P., S. P. Staal, and J. A. Hoch. 1973. Phenotypes of pleiotropic-negative sporulation mutants of Bacillus subtilis. J. Bacteriol. 115:1063-1070.
3. Cangelosi, G. A., E. A. Best, G. Martinetti, and E. W. Nester. 1991. Genetic analysis of Agrobacterium, p. 384-397, Methods Enzymol., vol. 204. Academic Press.
4. Caparon, M. G., J. R. Scott, and H. M. Jeffrey. 1991. Genetic manipulation of pathogenic streptococci, p. 556-586, Methods Enzymol., vol. 204. Academic Press.
5. Cutting, S. M., and C. R. Harwood. 1990. Molecular biological methods for Bacillus. John Wiley & Sons Ltd., Chichester, England.
6. Dalton, T. L., R. I. Hobb, and J. R. Scott. 2006. Analysis of the role of CovR and CovS in the dissemination of Streptococcus pyogenes in invasive skin disease. Microb. Pathog. 40:221-7.
7. Hamilton, R. H., and M. Z. Fall. 1971. Loss of tumor-initiating ability in Agrobacterium tumefaciens by incubation at high temperature. Experientia 27:229-&.
8. Komeili, A., Z. Li, D. K. Newman, and G. J. Jensen. 2006. Magnetosomes are cell membrane invaginations organized by the actin-like protein MamK. Science 311:242-5.
9. Lynch, S. A., S. K. Desai, H. K. Sajja, and J. P. Gallivan. 2007. A high-throughput screen for synthetic riboswitches reveals mechanistic insights into their function. Chem. Biol. 14:173-84.
10. Lynch, S. A., and J. P. Gallivan. 2009. A flow cytometry-based screen for synthetic riboswitches. Nucl. Acids Res. 37:184-92.
11. Ma, J., A. Campbell, and S. Karlin. 2002. Correlations between Shine-Dalgarno sequences and gene features such as predicted expression levels and operon structures. J. Bacteriol. 184:5733-5745.
12. Matsunaga, T., T. Sakaguchi, and F. Tadokoro. 1991. Magnetite formation by a magnetic bacterium capable of growing aerobically. Appl. Microbiol. Biotechnol. 35:651-655.
13. Miller, J. H. 1972. p. 352-355, Experiments in molecular genetics. Cold Spring Harbor Laboratory, Cold Spring Harbor, N.Y.
14. Murat, D., A. Quinlan, H. Vali, and A. Komeili. 2010. Comprehensive genetic dissection of the magnetosome gene island reveals the step-wise assembly of a prokaryotic organelle. Proc. Natl. Acad. Sci. USA 107:5593-5598.
15. Snapper, S. B., R. E. Melton, S. Mustafa, T. Kieser, and J. W. R. Jacobs. 1990. Isolation and characterization of efficient plasmid transformation mutants of Mycobacterium smegmatis. Mol. Microbiol. 4:1911-1919.
16. Topp, S., and J. P. Gallivan. 2008. Random walks to synthetic riboswitches—a high-throughput selection based on cell motility. Chem Bio Chem 9:210-3.
17. Vaneechoutte, M., D. M. Young, L. N. Ornston, T. De Baere, A. Nemec, T. Van Der Reijden, E. Carr, I. Tjernberg, and L. Dijkshoorn. 2006. Naturally transformable Acinetobacter sp. strain ADP1 belongs to the newly described species Acinetobacter baylyi. Appl. Environ. Microbiol. 72:932-936.
18. Zuker, M. 2003. Mfold web server for nucleic acid folding and hybridization prediction. Nucl. Acids Res. 31:3406-15.

### Example 2

### Use of a Riboswitch-Based Inducible Expression Platform in Mycobacteria

### Materials and Methods

### Strains and Reagents

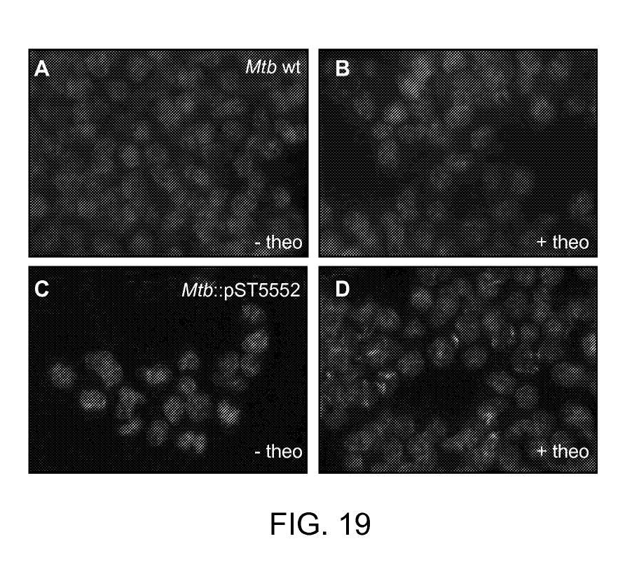

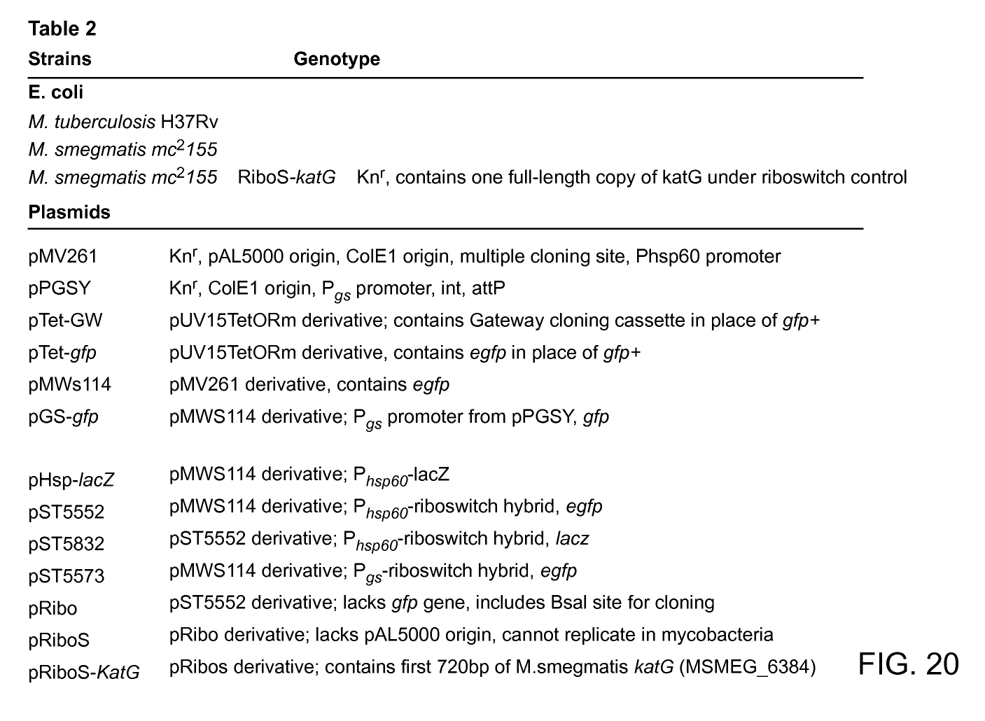

Theophylline, Tween 80, and 2-nitrophenyl β-D-galactopyranoside were purchased from Sigma. M. smegmatis mc²155 and M. tuberculosis H37Rv strains were used for all experiments below (see FIG. 20, which provides Table 2). Growth media 7H9 and 7H11 and OADC supplement were obtained from BD Biosciences. The growth medium was 7H9 (liquid) or 7H11 (solid) with 0.5% glycerol and 0.05% Tween-80 plus 0.5% glucose for M. smegmatis or 10% OADC for Mtb. The 7H11 solid growth medium for Mtb did not contain Tween. All kanamycin (Kn) concentrations are in units of μg/mL. PfuUltra DNA polymerase (Stratagene) was used for all site-directed mutagenesis and cloning according to the manufacturer's instructions plus the addition of 3% dimethylsulfoxide (DMSO). Taq Master Mix (Promega) was used for PCR verification of the recombinant strain. RAW 264.7 murine macrophages (ATCC #TIB-71) were cultured in RAW media (RPMI-1640 plus L-glutamine and 10% fetal bovine serum) unless otherwise noted.

### Design and Construction of Riboswitch-Reporter Plasmids

See FIG. 20 (Table 2) for a summary of all constructs used in this study. The egfp gene (hereafter referred to as gfp) encoding the fluorescence-enhancing mutations F64L and S65T was subcloned from pEGFP-N1 (Clontech) into the mycobacterial shuttle plasmid pMV261 using the EcoRI and HindIII restriction sites to create pMWS114. The construction of pST5552 was previously described in (21). Briefly, the 5′ untranslated region (168 bp upstream of the start codon) is predicted by the mFold program to be highly structured (34) and could interfere with riboswitch function. This portion of the M. bovis BCG hsp60 promoter (Pₕₛₚ₆₀) was removed and replaced by assembly PCR methods with a theophylline riboswitch. A positive control for constitutive β-galactosidase expression was constructed by subcloning the lacZ gene from pSKD345.1 (28) into pMWS114 using MscI and HindIII restriction sites to create pHsp60-lacZ.

The M. smegmatis glutamine synthetase promoter (P_{gs}) was subcloned from pPGSY into pMWS114 using XbaI and PstI restriction sites to create pGS-gfp, and the RBS was replaced with the theophylline riboswitch as above to create pST5573. Additional P_{gs}-riboswitch constructs incorporating four other theophylline riboswitch variants were assembled similarly (21).

Construction of pTet-gfp

The Tet-controlled GFP reporter construct pUV15tetORm contains gfp+, a variant that encodes folding mutations (F99S, M153T, V163A) not present in the egfp gene used in the above constructs (13). To create a Tet-controlled reporter construct that could be directly compared to the riboswitch system, the egfp gene was subcloned from pMWS114 using PacI and EcoRV into pTet-GW (gift from Prof. Christopher Sassetti, University of Massachusetts Medical School) to generate pTet-gfp.

### GFP Fluorescence Assay

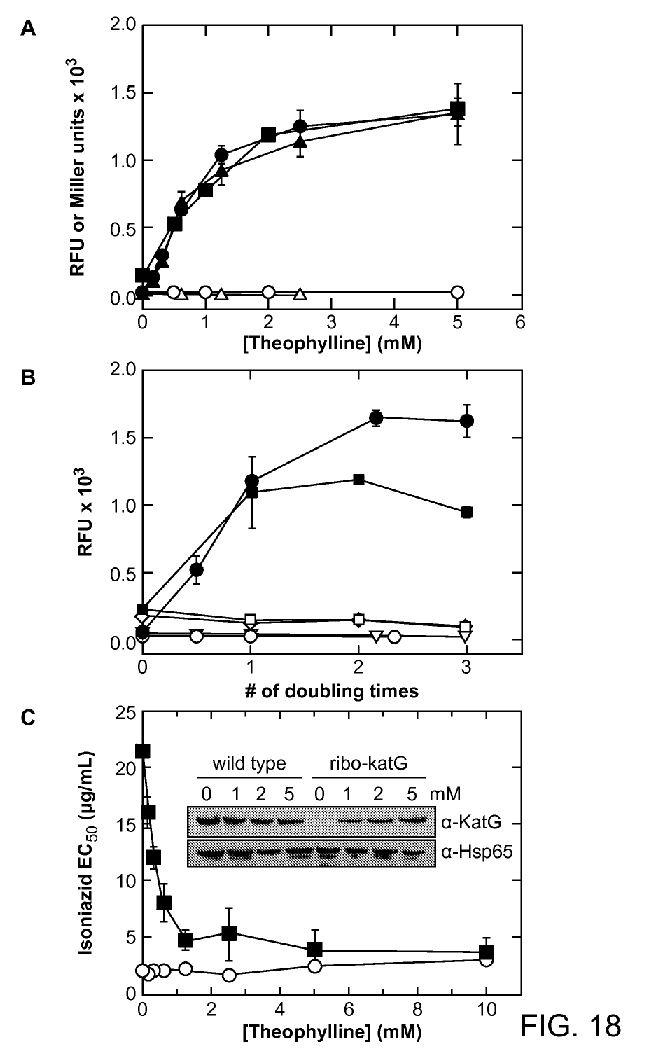

GFP assays for M. smegmatis transformed with pMV261, pMWS114, pST5552, pST5573, pTet-gfp, and pGS-gfp were performed as reported (21). Briefly, for dose response curves, cultures were grown from early- to late-log phase (optical density at 600 nm [OD₆₀₀] of 0.2 or 0.3 to ˜1) over two doubling times (6 h) in selective media containing 0-5 mM theophylline. Induction time courses were performed similarly, except cultures were induced with 2 mM theophylline at OD₆₀₀ of 0.1. At each time point, the OD₆₀₀ was measured and 1-mL aliquots pelleted and stored at −80° C. until measurement. Each cell pellet was resuspended in 200 μL PBS containing 0.05% Tween-80 and aliquoted into 96-well plates. Emission was measured at 510 nm with excitation at 450 nm and a 495-nm high-pass cutoff filter in a Gemini XPS fluorescence microplate reader (Molecular Devices Corporation). Growth inhibition was observed only at the highest concentrations of theophylline used in these assays (>5 mM; FIG. 18).

The GFP reporter constructs were also electroporated into Mtb and selected on 7H11/Kn(25). Growth, induction, and GFP assays were performed as described above except that cells were resuspended and incubated at room temperature for 1 h in 200 μL phosphate-buffered 10% formalin containing 0.05% Tween-80 prior to fluorescence measurement. All GFP data are reported as relative fluorescence (RFU) normalized by the OD₆₀₀ for each sample, and each data point represents the average of three replicates.

### β-Galactosidase Activity Assay

M. smegmatis transformed with pMV261, pHsp-lacZ or pST5832 was grown and harvested as for the GFP assay. Cell pellets were resuspended in 1 mL Z buffer (60 mM Na₂HPO₄, 40 mM NaH₂PO₄, 10 mM KCl, 1 mM MgSO₄, 50 mM (3-mercaptoethanol, pH 7.0). Cells were lysed with two pulses of 20 s each at power level 0.5 with a tip sonicator (Sonicator 3000, Misonix, Inc.). At t=0 min, 50 μL 2-nitrophenyl β-D-galactopyranoside (4 mg/mL in Z buffer) was added to 200 μL cell lysate. Reactions were incubated at 30° C. until yellow color was visible (˜10 min.). After recording the time and stopping the reaction with 125 μL 1 M sodium bicarbonate, cell debris was pelleted and the final OD₄₂₀ was recorded. Substrate turnover is reported in Miller units: (OD₄₂₀×1000)/(OD₆₀₀×r×n time in min).

Construction of M. smegmatis with KatG Under Riboswitch Control

pRibo was created by PCR-based site-directed mutagenesis of pST5552 to delete the gfp gene and simultaneously insert a BsaI restriction site immediately following the start codon to generate pRibo. The mycobacterial origin of replication (oriM) between MluI and NotI restriction sites was removed in a second mutagenesis step to create the plasmid pRiboS, which cannot replicate in mycobacteria. The first 720 bp of katG (MSMEG_{—}6384; GeneID 4536370) plus the stop codon TAA were PCR-amplified from M. smegmatis genomic DNA (35) and ligated into pRiboS using BsaI to create pRiboS-katG. Approximately 2 μg of this construct was UV-irradiated with 100 mJ cm⁻² to promote recombination and electroporated into M. smegmatis (36). Single recombinants were selected with Kn(20) and grown up in selective medium. Genomic DNA was extracted as above, and recombination at the katG locus was verified by PCR. In the resulting M. smegmatis strain RiboS-katG, the insertion of the entire plasmid at the katG locus by a single homologous recombination event results one full-length katG copy under control of the hybrid Pₕₛₚ₆₀-riboswitch promoter.

### Isoniazid Antibiotic Resistance Assay

M. smegmatis wild-type and RiboS-katG strains were grown to late-log phase and diluted to an OD₆₀₀ of 0.1 in 0-100 μg/mL isoniazid and 0-10 mM theophylline in 96-well plates containing 200 μL of culture per well. Plates were incubated without shaking at 37° C., and the final OD₆₀₀ recorded after 24 h. For each theophylline concentration, the OD₆₀₀ as a function of isoniazid concentration was fit to a single exponential using Kaleidagraph (Synergy Software). The half-maximum effective concentration of isoniazid (EC₅₀) at each theophylline concentration was averaged across at least three independent experiments for each strain.

### Anti-KatG Immunoblot

M. smegmatis wild-type and RiboS-katG strains were grown to late-log phase and diluted to OD₆₀₀ of 0.3 in 10 mL 7H9 with 0, 0.5, 1, or 2 mM theophylline. After 6 h incubation, cells were pelleted, resuspended in phosphate buffered saline (PBS)+5 μg/mL DNase, and lysed by tip sonication (10 s on, 10 s off, 2 min total processing time). Cleared whole-cell lysate (45 μg per sample) was separated by sodium dodecyl sulfate-polyacrylamide gel electrophoresis (SDS-PAGE) (10% Criterion gel, Bio-Rad Laboratories), blotted, and probed with mouse anti-KatG antibody (TB Vaccine Testing and Research Materials Contract HHSN266200400091c, Colorado State University) and an anti-Mtb GroEL2 (BDI1577; ab20519 from Abam), which cross-reacts with M. smegmatis (Msmeg) GroEL. Bands were visualized by enhanced chemiluminescence (SuperSignal West Pico Chemiluminescent Substrate, Pierce).

### Macrophage Infection and Microscopy

The RAW 264.7 murine macrophages were seeded on 22 mm×22 mm sterile glass coverslips in 6-well plates at 3×10⁵ cells per well and grown in 2 mL per well of RAW media for 1 day. Mtb wild type, Mtb::pMWS114, Mtb::pGS-gfp, Mtb::pST5552, and Mtb::pST5573 were grown to late-log phase. An aliquot of each culture was spun at 500×g for 5 min to remove cell clumps, and the supernatant was transferred to a fresh tube and spun at 3500×g for 5 min. The resulting cell pellet was washed twice with equal volumes of PBS and then diluted in the appropriate volume of RAW media (with 10% horse serum instead of fetal calf serum) for a multiplicity of infection (MOI) of 5 bacteria per macrophage. The macrophages were incubated in the resulting bacterial suspension for 4 h and washed 3 times with PBS. Infected macrophages were allowed to recover in RAW media until replacement at 24 h post infection with fresh media containing 0 or 0.5 mM theophylline. After an additional day (48 h post infection), macrophages were washed with 3 times with PBS and then fixed in phosphate-buffered 10% formalin for 1 h. Coverslips were mounted on glass slides with 20 μL VectaShield mounting medium plus 4′,6-diamidino-2-phenylindole (DAPI) stain (Vector Laboratories). A Zeiss Axiovert 200M inverted microscope with a 100×1.3 numerical aperture Plan-Apochromat oil immersion lens and filters appropriate for detecting GFP and DAPI fluorescence was used for imaging. Image stacks were acquired using a CoolSNAP HQ camera (Roper Scientific) and digitally deconvolved using the nearest-neighbors algorithm in Slidebook (Intelligent Imaging Solutions). Final images were generated by z-projection of between 30 and 40 frames at 0.34 μm separation and are representative of three biological replicates for each condition.

### Results

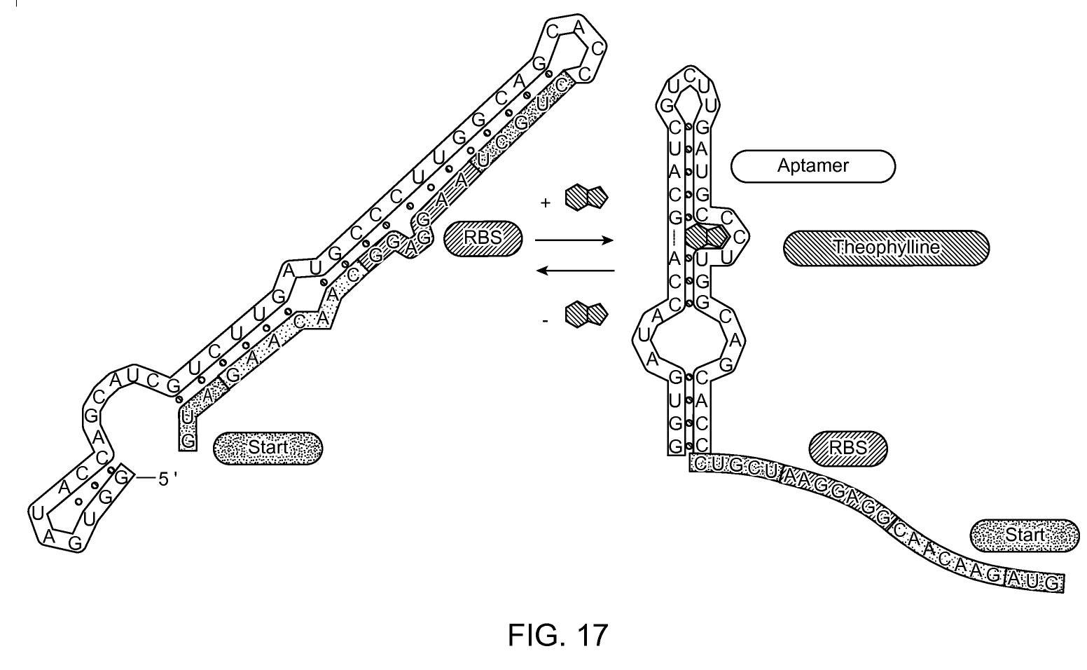

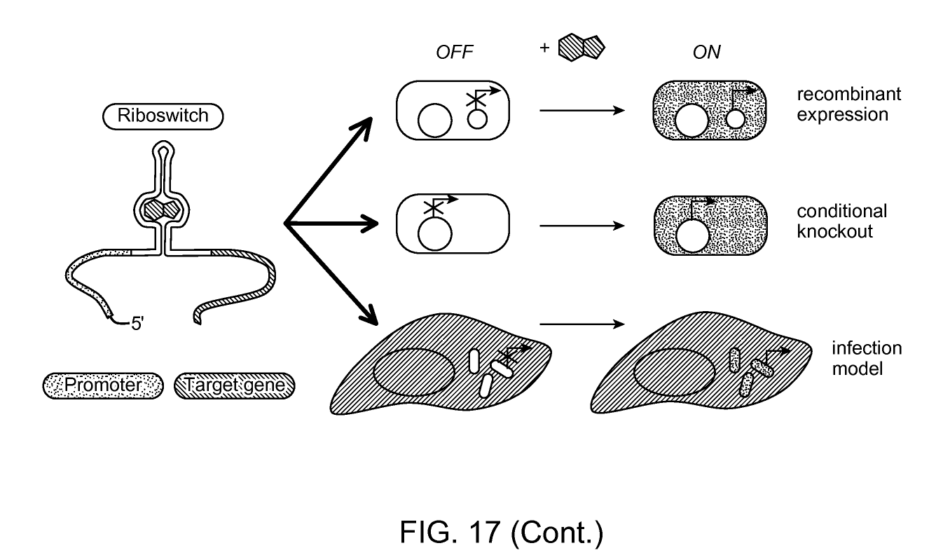

The theophylline riboswitch discussed in Example 1 can control gene expression in a range of Gram-negative and Gram-positive bacteria, including M. smegmatis. The riboswitch, which contains an RNA aptamer sequence that binds theophylline, can be combined with different transcriptional regulatory elements to generate inducible expression systems. In the absence of theophylline the mRNA transcript adopts a fold that sequesters the ribosome binding site (RBS) and prevents protein translation (FIG. 17). Upon theophylline binding, the mRNA adopts a conformation that liberates the RBS and allows protein synthesis. The entire riboswitch-based regulatory machinery is contained within a single ˜60-bp nucleic acid sequence. Significantly, no regulator proteins are involved in the induction mechanism, making the system easy to both modify and move.

This Example provides examples of applications of a riboswitch-based expression platform that exhibits a theophylline dose-dependent response in both M. smegmatis and M. tuberculosis. The versatility and modularity of this system were demonstrated by screening a panel of riboswitch variants in combination with two different mycobacterial promoters. Notably, the dose and time response profiles of the most successful synthetic promoter-riboswitch combination compare favorably to those of existing gene regulation mycobacterial systems. This platform was used to modulate the drug resistance phenotype of a conditional gene knockout in M. smegmatis and to induce Mtb gene expression during macrophage infection (FIG. 18B). Together these data illustrate that the inducible riboswitch system is a highly versatile and tunable platform for controlling gene expression in mycobacteria.

FIG. 17.

Design and applications of the riboswitch-based gene regulation platform for mycobacteria. A synthetic theophylline-responsive riboswitch adopts a fold that sequesters the ribosome binding site (RBS). In the presence of theophylline, the riboswitch adopts a conformation in which the RNA aptamer is bound to theophylline and the RBS is released able to promote protein translation. The riboswitch is combined with a mycobacterial transcriptional promoter and a downstream target gene to generate inducible gene regulation for a range of applications. The sequence depicted is for riboswitch E*.

### Design and Characterization of Riboswitch-Controlled Gene Expression in Mycobacteria

Riboswitches are described in Example 1. A riboswitch was combined with a strong mycobacterial promoter, the widely used constitutive promoter Pₕₛₚ₆₀, which is derived from the upstream region of the M. bovis BCG hsp60 gene (22).

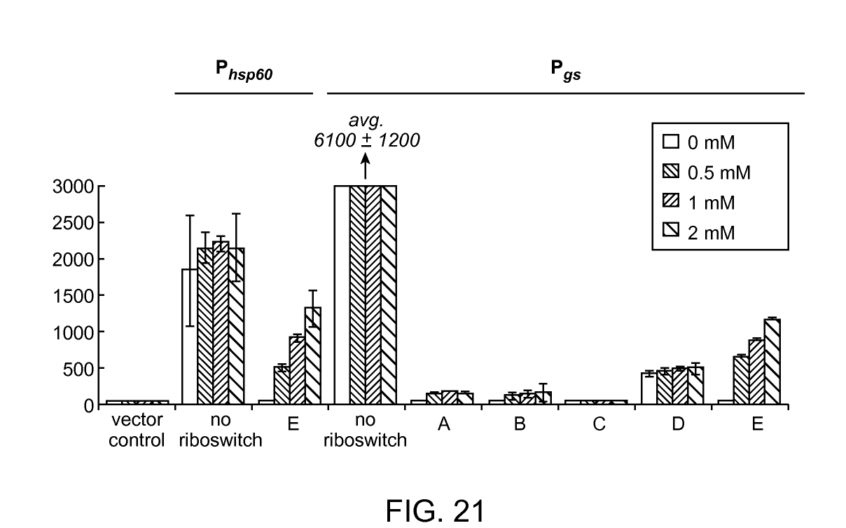

To test the modularity of the two-part promoter-riboswitch strategy in mycobacteria, a library of constructs was created by combining the set of five riboswitches with P_{gs}, a promoter derived from the upstream region of the M. tuberculosis glnA1 glutamine synthase gene (23). The ability of these new constructs to regulate GFP expression in the presence of 0-5 mM theophylline was measured in a whole-cell fluorescence assay. Although P_{gs} is a stronger promoter than Pₕₛₚ₆₀, it was found that riboswitch-P_{gs} constructs behave similarly to those derived from Pₕₛₚ₆₀ (FIG. 21). The maximal dose-dependent response was observed with riboswitch E* (21). The activation ratio, defined as the response at 2 mM vs. 0 mM theophylline, was 36±2.2 for P_{gs} and 54±11 for Pₕₛₚ₆₀, indicating robust response to theophylline.

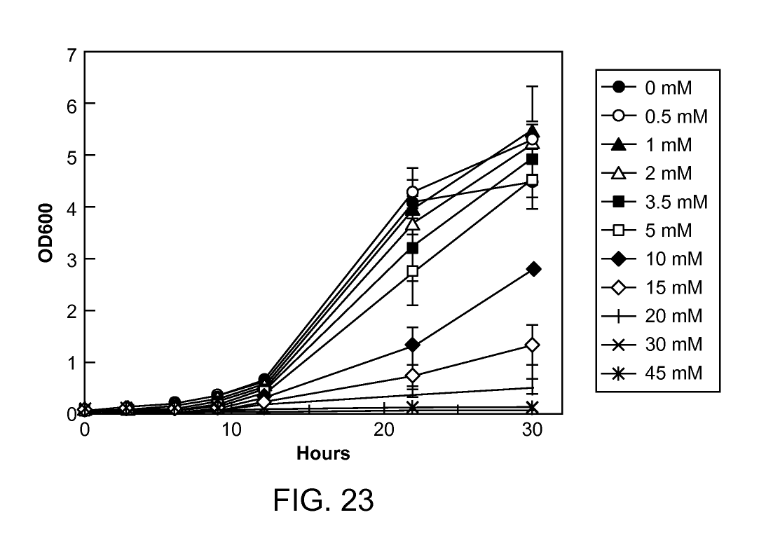

Since the Pₕₛₚ₆₀-riboE* pairing yielded the most robust response, it was tested in several applications of particular relevance to mycobacteria. To assess the generality of riboswitch response across different target genes, two Pₕₛₚ₆₀-riboE*-controlled constructs were used, which were designed to express either β-galactosidase or GFP and compared their responses M. smegmatis. For both, dose-dependent induction by theophylline was observed with maximum reporter expression at ˜2 mM theophylline (FIG. 18A), whereas significant growth attenuation was only observed at >2-fold higher concentrations of theophylline (FIG. 23). Also, the activation ratio for the two reporter genes was similar (89±12 for β-Gal and 65±8 for GFP at 2 mM theophylline), suggesting that riboswitch control functions independently of the target gene sequence (FIG. 18C).

Using the GFP reporter construct, a similar dose and time response was observed in Mtb as in M. smegmatis (FIG. 18A, 18B). For the vector-transformed and wild-type negative controls, higher signal was observed from Mtb than M. smegmatis in the GFP assay, possibly due to greater scattering from Mtb cells. This resulted in lower apparent activation ratios (FIG. 18C), but the overall dose response and maximum GFP expression level is equivalent between the two bacteria. Also, maximal GFP expression was observed at ˜2 mM theophylline after two doubling times for both bacteria. Overall, the activation ratio and time response of the inducible riboswitch system compare favorably with the ˜100-fold activation ratios and 2-day maximum induction times observed in the nitrile-inducible and Tn10-derived Tet systems (13, 19). In a direct comparison between the riboswitch and Tet systems in M. smegmatis, the activation ratio for GFP was identical (69±3 for the riboswitch vs. 72±5 for Tet).

These data confirm that the theophylline inducible riboswitch can regulate gene expression in both the model organism M. smegmatis and the pathogen Mtb, suggesting that the mechanism of riboswitch induction is mycobacterial species-independent. The similarity in responses between the two species also shows that M. smegmatis can serve as a host for screening further iterations of riboswitch-based mycobacterial gene regulation.

FIGS. 18A-C.

Verification of theophylline-induced gene regulation in M. smegmatis (Msmeg) and M. tuberculosis (Mtb). (A) Pₕₛₚ₆₀-riboE*-controlled GFP fluorescence in Msmeg (filled circles) and Mtb (filled squares) and β-galactosidase activity in Msmeg (filled triangles) in response to incubation in 0-5 mM theophylline for 6 h. Msmeg vector controls for GFP fluorescence and β-galactosidase activity are shown as open circles and triangles. Data are presented as relative fluorescence (RFU) for GFP and Miller units for β-galactosidase, and as mean±SEM of three independent experiments. (B) GFP expression as a function of time in 0 mM (open) or 2 mM (filled) theophylline for Msmeg (circles) and Mtb (squares). Msmeg vector and Mtb wild type controls are shown as triangles and diamonds. Doubling times for Msmeg and Mtb are approximately 3 and 24 h, respectively. Data are presented as mean±SEM of three independent experiments. (C) Isoniazid EC₅₀ for Msmeg wild type (open circles) and RiboS-katG (filled squares) in response to 0-10 mM theophylline. Data are presented as mean±SEM of three independent experiments. (inset) Anti-KatG immunoblot for Msmeg wild type and RiboS-katG strains grown in 0-5 mM theophylline for 6 h. An immunoblot against Hsp65 (GroEL2) is shown as a loading control. Each immunoblot is representative of two independent experiments

Theophylline-Dependent Knockout of KatG in M. smegmatis

Chromosomal gene knockouts are commonly used to examine gene function or determine gene essentially; conditional gene knockouts afford the additional power of inducing expression or repression at a defined phase of growth or infection. To assess the ability of RiboMyc to control theophylline-dependent conditional knockout mutants, M. smegmatis katG (MSMEG_{—}6384), a homologue of Mtb katG (Rv1908c), which encodes a catalase-peroxidase that converts the antibiotic prodrug isoniazid into its active form (24), was targeted. A homologous recombinant strain was generated, in which katG is under RiboMyc control. As predicted, the enzyme KatG is not expressed in this strain in the absence of theophylline, resulting in an isoniazid resistance phenotype (FIG. 18C). In the presence of increasing theophylline concentrations, the EC₅₀ decreases, effectively restoring wild-type susceptibility to the drug. The theophylline-dependent induction of KatG expression was further verified by immunoblot; importantly, no KatG was detected in the absence of theophylline, indicating efficient repression (FIG. 18C, inset). Addition of 2-5 mM theophylline produced sufficient KatG to restore the wild-type phenotype, confirming the utility of the riboswitch for creating conditional gene knockouts.

### Theophylline-Dependent Expression in Mtb in a Macrophage Infection Model

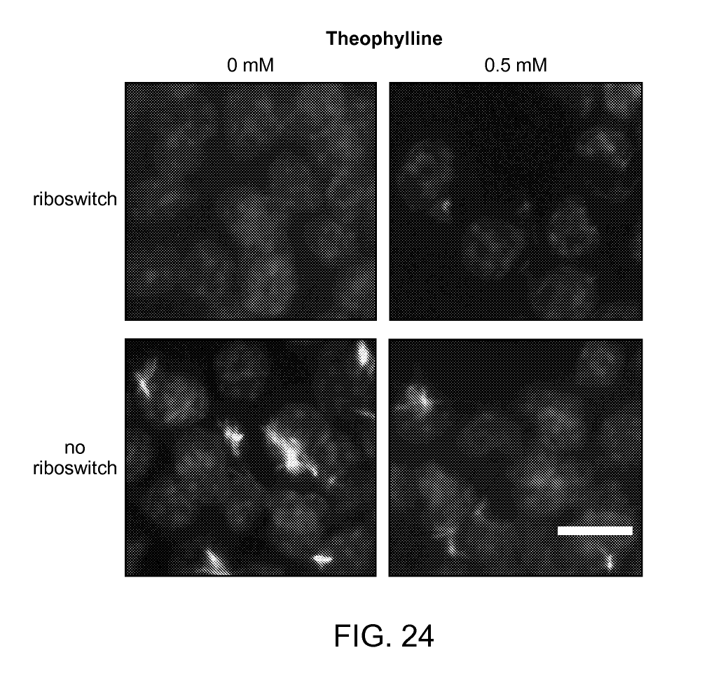

Gene expression in an intracellular pathogen such as Mtb is often regulated in response to changes in the host environment, such as internalization by macrophages (25). The ability to modulate expression levels during infection is critical to determining how specific genes affect bacterial survival and disease progression in the host. The RiboMyc system was tested in a macrophage-based infection model. The murine macrophage-like RAW 264.7 cell line was infected with Mtb harboring the riboswitch-GFP construct for 24 hours and induced with theophylline for an additional 24 hours. GFP fluorescence was observed from intracellular Mtb containing the riboswitch construct only in the presence of theophylline (FIG. 19). Thus, the riboswitch affords precise control over bacterial gene expression within host macrophages. Furthermore, the Pₕₛₚ₆₀- and P_{gs}-derived riboswitch constructs both exhibited theophylline-dependent responses in macrophages (FIG. 24). These data both validate the modularity of the RiboMyc platform and demonstrate the consistency of its response across in vitro and cell-based applications.

FIG. 19.

Demonstration of riboswitch-controlled Mtb gene expression in a macrophage infection model. RAW 264.7 murine macrophages infected with (A) Mtb::pST5552, (B) Mtb::pMWS114 or (C) Mtb wild type and induced with 0 mM (−theo) or 0.5 mM (+theo) theophylline for 24 h. Overlaid fluorescence signals from DAPI and GFP channels show nuclei (blue) and GFP-expressing bacteria (green). Theophylline-dependent induction of GFP expression is observed only in macrophages infected with Mtb harboring the riboswitch-gfp construct pST5552. Scale bar represents 10 μm.

FIG. 21.

P_{gs}-derived riboswitch expression systems behave similarly to those derived from Pₕₛₚ₆₀. Theophylline dose response of GFP expression from the promoter P_{gs} either alone (“no riboswitch”) or in combination with theophylline riboswitch variants A-E* (See Table 1 of Example 1). Response from the promoter Pₕₛₚ₆₀ alone and in combination with riboswitch E* is shown for comparison.

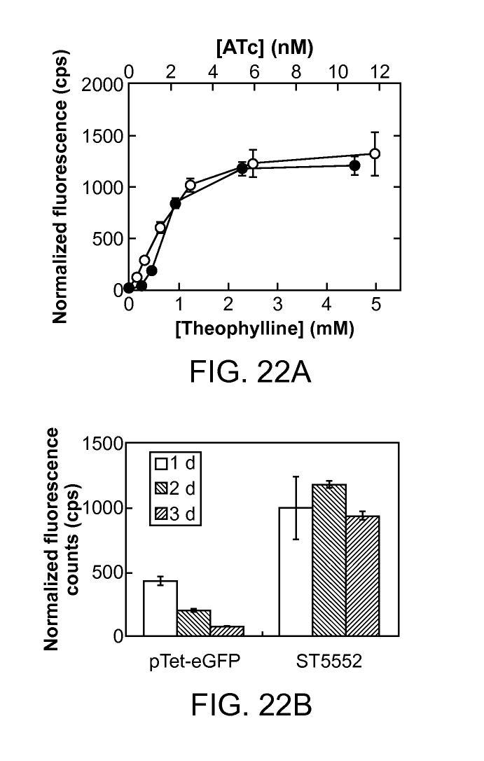

FIGS. 22A and 22B.

Theophylline-dependent induction of GFP or (β-galactosidase expression. Theophylline dose response of GFP (filled circles) and β-galactosidase (open circles) expression under riboswitch control in M. smegmatis.

FIG. 23.

M. smegmatis growth as a function of theophylline concentration. Growth curves (OD₆₀₀) for wild-type M. smegmatis in increasing concentrations of theophylline. Approximate minimum inhibitory concentration (MIC) is 30 mM.

FIG. 24.

Inducible Mtb gene expression in a macrophage infection model from a riboswitch based on the glutamine synthase promoter P_{gs}. RAW 264.7 murine macrophages infected with Mtb::pST5573 (“riboswitch”) or Mtb::pGS-gfp (“no riboswitch”) and treated with 0 mM or 0.5 mM theophylline for 24 h. Overlaid fluorescence signals from DAPI and GFP channels show nuclei (blue) and GFP-expressing bacteria (green). Scale bar represents 10 μm.

### REFERENCES

1. Balbás, P. & Lorence, A. eds. (2004) Recombinant Gene Expression: Reviews and Protocols (Humana Press, Totowa, N.J.).
2. Judson, N. & Mekalanos, J. J. (2000) Transposon-based approaches to identify essential bacterial genes Trends Microbiol. 8: 521-526.
3. Miesel, L., Greene, J., & Black, T. A. (2003) Genetic strategies for antibacterial drug discovery Nat Rev Genet 4: 442-456.
4. Payne, D. J., Gwynn, M. N., Holmes, D. J., & Pompliano, D. L. (2007) Drugs for bad bugs: confronting the challenges of antibacterial discovery Nat Rev Drug Discov 6: 29-40.
5. Sassetti, C. M. (2008) Inducible Expression Systems for Mycobacteria in Mycobacteria Protocols, eds. Parish, T. & Brown, A. C. (Humana Press, Totowa, N.J.), pp. 255-264.
6. Parish, T., Mahenthiralingam, E., Draper, P., Davis, E. O., & Colston, E. O. (1997) Regulation of the inducible acetamidase gene of Mycobacterium smegmatis Microbiology 143: 2267-2276.
7. Parish, T. & Stoker, N. G. (1997) Development and use of a conditional antisense mutagenesis system in mycobacteria FEMS Microbiol. Lett. 154: 151-157.
8. James, A. T., Tanya, P., Warwick, J. B., & Brigitte, G. (1998) An inducible expression system permitting the efficient purification of a recombinant antigen from Mycobacterium smegmatis FEMS Microbiol. Lett. 167: 151-156.
9. Brown, A. C. & Parish, T. (2006) Instability of the acetamide-inducible expression vector pJAM2 in Mycobacterium tuberculosis Plasmid 55: 81-86.
10. Blokpoel, M. C. J., Murphy, H. N., O'Toole, R., Wiles, S., Runn, E. S. C., Stewart, G. R., Young, D. B., & Robertson, B. D. (2005) Tetracycline-inducible gene regulation in mycobacteria Nucl. Acids Res. 33: e22-.
11. Hernandez-Abanto, S., Woolwine, S., Jain, S., & Bishai, W. (2006) Tetracycline-inducible gene expression in mycobacteria within an animal host using modified Streptomyces tcp830 regulatory elements Arch. Microbiol. 186: 459-464.
12. Carroll, P., Muttucumaru, D. G. N., & Parish, T. (2005) Use of a Tetracycline-Inducible System for Conditional Expression in Mycobacterium tuberculosis and Mycobacterium smegmatis Appl. Environ. Microbiol. 71: 3077-3084.
13. Ehrt, S., Guo, X. V., Hickey, C. M., Ryou, M., Monteleone, M., Riley, L. W., & Schnappinger, D. (2005) Controlling gene expression in mycobacteria with anhydrotetracycline and Tet repressor Nucl. Acids Res. 33: e21-.
14. Gandotra, S., Schnappinger, D., Monteleone, M., Hillen, W., & Ehrt, S. (2007) In vivo gene silencing identifies the Mycobacterium tuberculosis proteasome as essential for the bacteria to persist in mice Nat. Med. 13: 1515-1520.
15. Klotzsche, M., Ehrt, S., & Schnappinger, D. (2009) Improved tetracycline repressors for gene silencing in mycobacteria Nucl. Acids Res. 37: 1778-1788.
16. Paul, C., Amanda, C. B., Anna, R. H., & Tanya, P. (2007) Expression of Mycobacterium tuberculosis Rv1991c using an arabinose-inducible promoter demonstrates its role as a toxin FEMS Microbiol. Lett. 274: 73-82.
17. Kaur, P., Agarwal, S., & Datta, S. (2009) Delineating Bacteriostatic and Bactericidal Targets in Mycobacteria Using IPTG Inducible Antisense Expression PLoS ONE 4: e5923.
18. Ford, F., Crosta, A., & Ghisotti, D. (2009) Pristinamycin-inducible gene regulation in mycobacteria J. Biotechnol. 140: 270-277.
19. Pandey, A. K., Raman, S., Proff, R., Joshi, S., Kang, C.-M., Rubin, E. J., Husson, R. N., & Sassetti, C. M. (2009) Nitrile-inducible gene expression in mycobacteria Tuberculosis 89: 12-16.
20. Boldrin, F., Casonato, S., Dainese, E., Sala, C., Dhar, N., Palu, G., Riccardi, G., Cole, S. T., & Manganelli, R. (2010) Development of a repressible mycobacterial promoter system based on two transcriptional repressors Nucl. Acids Res.: gkq235.
21. Topp, S., Reynoso, C. K., Seeliger, J. C., Goldlust, I. S., Desai, S. K., Murat, D., Shen, A., Komeili, A., Bertozzi, C. R., Scott, J. R., et al. Synthetic Riboswitches that Induce Gene Expression in Diverse Bacterial Species, submitted.
22. Stover, C. K., de la Cruz, V. F., Fuerst, T. R., Burlein, J. E., Benson, L. A., Bennett, L. T., Bansal, G. P., Young, J. F., Lee, M. H., Hatfull, G. F., et al. (1991) New use of BCG for recombinant vaccines Nature 351: 456-460.
23. Harth, G. & Horwitz, M. A. (1997) Expression and Efficient Export of Enzymatically Active Mycobacterium tuberculosis Glutamine Synthetase in Mycobacterium smegmatis and Evidence That the Information for Export is Contained within the Protein J. Biol. Chem. 272: 22728-22735.
24. Slayden, R. A. & Barry, r. C. E. (2000) The genetics and biochemistry of isoniazid resistance in Mycobacterium tuberculosis Microbes and Infection 2: 659-669.
25. Rohde, K. H., Abramovitch, R. B., & Russell, D. G. (2007) Mycobacterium tuberculosis Invasion of Macrophages: Linking Bacterial Gene Expression to Environmental Cues Cell Host & Microbe 2: 352-364.
26. Bonati, M., Latini, R., Tognoni, G., Young, J. F., & Garattini, S. (1984) Interspecies Comparison of In Vivo Caffeine Pharmacokinetics in Man, Monkey, Rabbit, Rat and Mouse Drug Metab. Rev. 15: 1355-1383.
27. Lynch, S. A., Desai, S. K., Sajja, H. K., & Gallivan, J. P. (2007) A high-throughput screen for synthetic riboswitches reveals mechanistic insights into their function Chem. Biol. 14: 173-184
28. Desai, S. K. & Gallivan, J. P. (2004) Genetic screens and selections for small molecules based on a synthetic riboswitch that activates protein translation J. Am. Chem. Soc. 126: 13247-13254.
29. Breaker, R. R. (2008) Complex Riboswitches Science 319: 1795-1797.
30. Roth, A. & Breaker, R. R. (2009) The Structural and Functional Diversity of Metabolite-Binding Riboswitches Annu. Rev. Biochem. 78: 305-334.
31. Dixon, N., Duncan, J. N., Geerlings, T., Dunstan, M. S., McCarthy, J. E. G., Leys, D., & Micklefield, J. (2010) Reengineering orthogonally selective riboswitches PNAS 107: 2830-2835.
32. Wayne, L. G. & Sohaskey, C. D. (2001) Nonreplicating Persistence of Mycobacterium tuberculosis Annu. Rev. Microbiol. 55: 139-163.
33. Joanna, C. B., Pauline, T. L., Linda, C. R., Ruth, A. M., & Ken, D. (2002) Evaluation of a nutrient starvation model of Mycobacterium tuberculosis persistence by gene and protein expression profiling Mol. Microbiol. 43: 717-731.
34. Zuker, M. (2003) Mfold web server for nucleic acid folding and hybridization prediction Nucl. Acids Res. 31: 3406-3415.
35. Larsen, M., Biermann, K. E., Tandberg, S., Hsu, T., & Jacobs Jr, W. R. (2007) Genetic Manipulation of Mycobacterium tuberculosis Current Protocols in Microbiology 10A: 2.1-2.21.
36. Hinds, J., Mahenthiralingam, E., Kempsell, K. E., Duncan, K., Stokes, R. W., Parish, T., & Stoker, N. G. (1999) Enhanced gene replacement in mycobacteria Microbiology 145: 519-527.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A synthetic theophylline-responsive translation regulator, wherein the synthetic translation regulator comprises, in order from 5′ to 3′:
a) a theophylline-binding aptamer;
b) a first linker of from 0 to 20 nucleotides in length;
c) a ribosome binding site; and
d) a second linker of from 0 to 20 nucleotides in length.

2. The synthetic translation regulator of claim 1, wherein the theophylline-binding aptamer comprises the sequence 5′-GGUGAUACCAGCAUCGUCUUGAUGCCCUUGGCAGCACC-3′ (SEQ ID NO:18).

3. The synthetic translation regulator of claim 1, wherein the ribosome binding site has a length of from 4 nucleotides to 10 nucleotides, and comprises the sequence AAGG.

4. The synthetic translation regulator of claim 1, wherein the ribosome binding site comprises a sequence selected from AGGGGGU, AAGGGG, AAGGG, AAGGU, AAGGAGGU, and AAGGAGG.

5. A gene expression cassette, the cassette comprising, in order from 5′ to 3′ and in operable linkage:
a) a promoter active in a bacterial cell;
b) a 5′ untranslated region; and
c) a synthetic translation regulator of claim 1.

6. The gene expression cassette of claim 5, wherein the 5′ UTR comprises the sequence 5′-ATACGACTCACTATA-3′ (SEQ ID NO:10).

7. The gene expression cassette of claim 5, wherein the promoter is an inducible promoter.

8. The gene expression cassette of claim 5, wherein the promoter is a constitutive promoter.

9. The gene expression cassette of claim 5, further comprising, 3′ of, and in operable linkage with, the synthetic translation regulator, a coding region comprising a 5′ ATG.

10. The gene expression cassette of claim 9, wherein the coding region comprises a nucleotide sequence encoding a therapeutic polypeptide, a regulatory polypeptide, a structural polypeptide, a secreted polypeptide, an enzyme, or a polypeptide that directly or indirectly produces a detectable signal.

11. A gene expression construct comprising the gene expression cassette of claim 5 inserted into a plasmid suitable for use in a bacterial cell.

12. The gene expression construct of claim 11, wherein the plasmid is a high copy number plasmid.

13. The gene expression construct of claim 11, wherein the plasmid is a low copy number plasmid.

14. The gene expression construct of claim 11, wherein the plasmid integrates into the chromosome of a bacterial host cell.

15. A genetically modified bacterium comprising the gene expression construct of claim 11.

16. The genetically modified bacterium of claim 15, wherein the bacterium is a Gram-negative bacterium.

17. The genetically modified bacterium of claim 15, wherein the bacterium is a Gram-positive bacterium.

18. The genetically modified bacterium of claim 15, wherein the bacterium is a human pathogen.

19. A method of modulating translation of a coding region in a bacterial cell, the method comprising contacting the cell with theophylline, wherein the cell is genetically modified with a nucleic acid comprising a synthetic translation regulator of claim 1 operably linked to the coding region, and wherein, in the presence of theophylline, translation of the coding region is increased, compared to the level of translation of the coding region in the absence of theophylline.

20. The method of claim 19, wherein the bacterium is a Gram-negative bacterium.

21. The method of claim 19, wherein the bacterium is a Gram-positive bacterium.

22. The method of claim 19, wherein the bacterium is a human pathogen.
